SEQUENCE LISTING (4 sequences)

SEQ ID NO: 1 - protein, 465 aa, human
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 2 - RNA, 19 nt, artificial sequence
  siRNA; synthetic construct
uuccaaaccggaugagugg

SEQ ID NO: 3 - RNA, 19 nt, artificial sequence
  siRNA; synthetic construct
gagcgggagaccuggagca

SEQ ID NO: 4 - RNA, 19 nt, artificial sequence
  siRNA; synthetic construct
cuccuggaacacuggcaac

(19) United States
(12) Patent Application Publication
(10) Pub. No.: US 2004/0247586 A1
(43) Pub. Date: Dec. 9, 2004

(54) Parkin-associated complex for protecting post-mitotic neurons from excitotoxicity and uses thereof

(76) Inventors: Asa Abeliovich, New York, NY (US); John Francois Staropoli, New York, NY (US)

Correspondence Address: Leslie Gladstone Restaino, 163 Madison Avenue, P.O. Box 1989, Morristown, NJ, 07962-1989, US

(21) Appl. No.: 10/783,780

(22) Filed: Feb. 20, 2004

Related U.S. Application Data
(60) Provisional application No. 60/452,331, filed on Mar. 5, 2003.

(51) Int. Cl. A61K 38/48; C12N 9/64

(52) U.S. Cl. 424/94.63; 435/226

(57) ABSTRACT

The present invention provides a parkin-associated complex. The present invention further provides methods for promoting ubiquitination of cyclin E in neurons, for decreasing cyclin E in neurons, for treating or preventing neurodegeneration in a subject, and for protecting neurons from excitotoxicity. Additionally, the present invention provides a therapeutic composition, and use of the therapeutic composition in an animal model. The present invention further provides a method for identifying an agent which interacts with a parkin-associated complex, agents identified by this method, and use of a parkin-associated agent to protect neurons from excitotoxicity. Additionally, the present invention provides methods for determining whether a subject has neurodegeneration, for assessing the efficacy of therapy to treat neurodegeneration in a subject, and for assessing the prognosis of a subject who has neurodegeneration. Finally, the present invention provides a kit for use in detecting neurodegeneration.

### RELATED APPLICATIONS

This application claims the benefit of U.S. Provisional Application Ser. No. 60/452,331, filed on Mar. 5, 2003, and entitled “A PARKIN-ASSOCIATED COMPLEX FOR PROTECTING POST-MITOTIC NEURONS FROM EXCITOTOXICITY AND USES THEREOF”, the contents of which are hereby incorporated by reference herein.

### STATEMENT OF GOVERNMENT INTEREST

This invention was made with government support under NIH Grant No. R21 NS43744-01. As such, the United States government has certain rights in this invention.

### BACKGROUND OF THE INVENTION

Parkinson's disease (PD) is a progressive, neurodegenerative disease, the symptoms of which include tremors, speech impediments, movement difficulties, and dementia. PD is pathologically characterized by loss, in the substantia nigra, of nerve cells containing dopamine. As a consequence, dopamine is deficient in Parkinson's patients. The cause of PD is unknown. However, recent studies indicate that two separate mutations in the gene coding for alpha-synuclein are responsible for certain rare familial forms of PD. More recent epidemiological studies indicate that parkin is also defective in a significant percentage of all familial PD. Deprenyl (selegiline) may slow progression of PD, if it is begun early in the disorder. There is also evidence that antioxidants, such as selenium and vitamin E, may be of some benefit. Nevertheless, there is still no known cure for PD.

Altered protein degradation through the ubiquitin proteasome pathway (UPP) has been hypothesized to underlie several neurodegenerative syndromes, including Parkinson's disease (PD) (Alves-Rodrigues et al., Ubiquitin, cellular inclusions and their role in neurodegeneration. Trends Neurosci., 21:516-20, 1998). Affected neurons in sporadic PD brain, particularly dopaminergic neurons in the substantia nigra, typically display ubiquitin-rich intracytoplasmic protein aggregates known as Lewy bodies (LBs) (Lang and Lozano, Parkinson's disease. First of two parts. N. Engl. J. Med., 339:1044-53, 1998). Additionally, a mutation in a ubiquitin carboxyl-terminal hydrolase gene, uch-L1, has been linked to a rare, autosomal dominant form of PD (Leroy et al., The ubiquitin pathway in Parkinson's disease. Nature, 395:451-52, 1998).

Further implicating the UPP in PD, mutations in a putative ubiquitin ligase gene, parkin, underlie an autosomal recessive, early-onset form of Parkinson's disease (ARPD) (Kitada et al., Mutations in the parkin gene cause autosomal recessive juvenile parkinsonism. Nature, 392:605-08, 1998). Parkin harbors two RING finger motifs at its carboxyl terminus. Such RING motifs are found in a number of E3 ubiquitin ligases (Joazeiro and Weissman, RING finger proteins: mediators of ubiquitin ligase activity. Cell, 102:549-52, 2000). Ubiquitin ligases control the specificity of substrate selection by the ubiquitination machinery, and collaborate with two other activities: a ubiquitin-activating enzyme (E1) and a ubiquitin-conjugating enzyme (E2) (Hershko et al., Components of ubiquitin-protein ligase system. Resolution, affinity purification, and role in protein breakdown. J. Biol. Chem., 258:8206-14, 1983). Polyubiquitin protein conjugates generated by this cascade are subsequently recognized and degraded by the 26S proteasome complex.

The parkin gene product appears to be associated with ubiquitin ligase activity (Imai et aL, Parkin suppresses unfolded protein stress-induced cell death through its E3 ubiquitin-protein ligase activity. J. Biol. Chem., 275(46):35661-664, 2000; Shimura et al., Familial Parkinson disease gene product, parkin, is a ubiquitin-protein ligase. Nat. Genet., 25:302-05, 2000; Zhang et al., Parkin functions as an E2-dependent ubiquitin- protein ligase and promotes the degradation of the synaptic vesicle-associated protein, CDCrel-1. Proc. Natl Acad. Sci. USA, 97:13354-359, 2000). Therefore, it is possible that mutations in parkin lead to a familial PD syndrome due to defective proteolysis (and consequent toxic accumulation) of parkin substrates.

Prior studies have described potential parkin substrates based on interaction assays. These proteins include CDCrel-1(Zhang et al., Parkin functions as an E2-dependent ubiquitin- protein ligase and promotes the degradation of the synaptic vesicle-associated protein, CDCrel-1. Proc. Natl Acad. Sci. USA, 97:13354-359, 2000), synphilin-1 (Chung et al., Parkin ubiquitinates the alpha-synuclein-interacting protein, synphilin-1 : implications for Lewy-body formation in Parkinson disease. Nat. Med., 7:1144-50, 2001), PAEL-R (Imai et al., An unfolded putative transmembrane polypeptide, which can lead to endoplasmic reticulum stress, is a substrate of Parkin. Cell, 105:891-02, 2001), and a modified form of α-synuclein (αSp22) (Shimura et al., Ubiquitination of a new form of alpha-synuclein by parkin from human brain: implications for Parkinson's disease. Science, 293:263-69, 2001). α-synuclein is of potential interest because mutations in this gene underlie rare autosomal dominant forms of PD (Polymeropoulos et al., Mutation in the alpha-synuclein gene identified in families with Parkinson's disease. Science, 276:2045-47, 1997). Furthermore, α-synuclein protein is enriched in LBs (Spillantini et al., Alpha-synuclein in Lewy bodies. Nature, 388:839-40, 1997).

All of the foregoing studies are based on the assumption that parkin-interacting proteins represent parkin substrates. However, a number of RING proteins have been shown to function within multiprotein ubiquitin ligase complexes in vivo (Joazeiro and Weissman, RING finger proteins: mediators of ubiquitin ligase activity. Cell, 102:549-52, 2000). Rbx family RING proteins (Kamura et al., Rbx1, a component of the VHL tumor suppressor complex and SCF ubiquitin ligase. Science, 284:657-61, 1999; Skowyra et al., Reconstitution of G1 cyclin ubiquitination with complexes containing SCFGrr1 and Rbx1. Science, 284:662-65, 1999) function within modular, multiprotein SCF complexes (for Skp1, Cullin, and F-box (Patton et al., Combinatorial control in ubiquitin-dependent proteolysis: don't Skp the F-box hypothesis. Trends Genet., 14:236-43, 1998; Skowyra et al., F-box proteins are receptors that recruit phosphorylated substrates to the SCF ubiquitin-ligase complex. Cell, 91:209-19, 1997). The Drosophila protein, Sina, and its mammalian homologue, Siah-1, similarly interact with SCF components, including the F-box/WD-repeat domain protein, Ebi, which serves to specify substrates for this complex (Dong et al., ebi regulates epidermal growth factor receptor signaling pathways in Drosophila. Genes Dev., 13:954-65, 1999; Matsuzawa and Reed, Siah-1, SIP, and Ebi collaborate in a novel pathway for beta-catenin degradation linked to p53 responses. Mol. Cell, 7:915-26, 2001).

Several lines of evidence support the notion that parkin functions within a multiprotein E3 complex. First, parkin derived from cerebral cortex migrates at a disproportionately large apparent molecular weight (670-418 kDa) on gel filtration chromatography. Second, a yeast 2-hybrid interaction screen for parkin-associated proteins indicated that the WD-repeat motif constitutes a parkin-interacting module, as 4 out of 10 isolated candidate clones harbored this motif. However, prior to the present invention, it was not known whether parkin interacts with identified SCF components, including F-box/WD-repeat domain proteins.

The role of cyclins is best characterized in cell-cycle regulation of mitotic cells. However, increased cyclin activity has been shown to trigger apoptosis of post-mitotic neurons (Copani et al., Activation of cell-cycle-associated proteins in neuronal death: a mandatory or dispensable path? Trends Neurosci., 24:25-31, 2001; Liu and Greene, Neuronal apoptosis at the G1/S cell cycle checkpoint. Cell Tissue Res., 305:217-28, 2001; Padmanabhan et al., Role of cell cycle regulatory proteins in cerebellar granule neuron apoptosis. J. Neurosci., 19:8747-56, 1999; Park et al., Cyclin-dependent kinases participate in death of neurons evoked by DNA-damaging agents. J. Cell Biol., 143:457-67, 1998), and cyclins have been shown to accumulate in post-mitotic neurons in response to pro-apoptotic stimuli, such as excitotoxicity.

### SUMMARY OF THE INVENTION

The inventors disclose herein that parkin associates with the previously-characterized ubiquitin ligase components, hSel-10 (also termed hCdc4, Archipelago, and Fbw7) (Koepp et al., Phosphorylation-dependent ubiquitination of cyclin E by the SCFFbw7 ubiquitin ligase. Science, 294:173-77, 2001; Moberg et al., Archipelago regulates Cyclin E levels in Drosophila and is mutated in human cancer cell lines. Nature, 413:311-16, 2001; Strohmaier et al., Human F-box protein hCdc4 targets cyclin E for proteolysis and is mutated in a breast cancer cell line. Nature, 413:316-22, 2001; Wu et al., SEL-10 is an inhibitor of notch signaling that targets notch for ubiquitin-mediated protein degradation. Mol. Cell Biol., 21:7403-15, 2001) and Cullin-1 (Cul-1), in an E3 complex that uses the E2 UbcH7 to ubiquitinate target proteins. Furthermore, the inventors identify cyclin E, an hSel-10-interacting protein, as a candidate target of this complex.

The inventors also show herein that parkin over-expression suppresses both. cyclin E accumulation and apoptosis in post-mitotic neurons exposed to kainic acid, an excitotoxin. Additionally, the inventors demonstrate that cyclin E levels are elevated in parkin-deficient primary neurons, including midbrain dopamine neurons, and parkin-deficient brain extracts. Cyclin E and additional cyclins, as well as cyclin-dependent kinases (CDKs), accumulate in a number of neurodegenerative disorders, including PD (Husseman et al., Mitotic activation: a convergent mechanism for a cohort of neurodegenerative diseases. Neurobiol. Aging, 21:815-28, 2000); Neystat et al., Expression of cyclin-dependent kinase 5 and its activator p35 in models of induced apoptotic death in neurons of the substantia nigra in vivo. J. Neurochem., 77:1611-25, 2001). Thus, the inventors hypothesize that parkin mutations lead to dopamine neuron loss in ARPD as a consequence of increased susceptibility of post-mitotic neurons to toxic insults.

Accordingly, in one aspect, the present invention provides a parkin-associated complex, comprising parkin, hSel-10, and cullin-1.

The present invention further provides a method for promoting ubiquitination of cyclin E in a post-mitotic neuron, by increasing activity of a parkin-associated complex in the neuron, wherein the parkin-associated complex comprises parkin, hSel-10, and cullin-1.

Additionally, the present invention provides a therapeutic composition, comprising: (a) a nucleic acid encoding a parkin-associated agent; (b) a lentiviral vector; and (c) optionally, a pharmaceutically-acceptable carrier; wherein the parkin-associated agent is selected from the group consisting of a parkin protein, a parkin mimetic, a modulator of parkin expression, and a modulator of parkin activity. Also provided are a method for treating neurodegeneration in a subject in need of treatment, by administering the therapeutic composition to the subject, and use of the therapeutic composition in an animal model of Parkinson's disease.

The present invention further provides a method for identifying an agent which interacts with a parkin-associated complex, comprising the steps of: (a) contacting a candidate agent with the complex, in the presence of cyclin E; and (b) assessing the ability of the candidate agent to enhance interaction between the complex and cyclin E; wherein the parkin-associated complex comprises parkin, hSel-10, and cullin-1. This method may further comprise the steps of: (c) contacting the candidate agent with at least one post-mitotic neuron containing cyclin E; and (d) determining if the agent has an effect on a cyclin-E-associated biological event in the at least one neuron. Also provided are agents identified by these methods, as well as methods for protecting at least one post-mitotic neuron from excitotoxicity, and for treating or preventing neurodegeneration in a subject, using these agents.

Furthermore, the present invention provides a method for decreasing cyclin E in at least one post-mitotic neuron, by contacting the at least one neuron with a parkin-associated agent, in an amount effective to decrease cyclin E in the neuron, wherein the parkin-associated agent is selected from the group consisting of a parkin protein, a parkin mimetic, a modulator of parkin expression, and a modulator of parkin activity.

The present invention further provides a method for protecting at least one post-mitotic neuron from excitotoxicity, by contacting the at least one neuron with a parkin-associated agent, in an amount effective to protect the neuron from excitotoxicity, wherein the parkin-associated agent is selected from the group consisting of a parkin protein, a parkin mimetic, a modulator of parkin expression, and a modulator of parkin activity. Also provided is use of the parkin-associated agent to protect a post-mitotic neuron from excitotoxicity, wherein the neuron is contacted with an amount of parkin-associated agent effective to protect the neuron from excitotoxicity.

Additionally, the present invention provides a method for determining whether a subject has neurodegeneration, by assaying a diagnostic sample of the subject for cyclin E, wherein detection of a cyclin E level elevated above normal is diagnostic of neurodegeneration in the subject. Also provided are methods for assessing the efficacy of therapy to treat neurodegeneration in a subject who has undergone or is undergoing treatment for neurodegeneration, and for assessing the prognosis of a subject who has neurodegeneration.

Finally, the present invention provides a kit for use in detecting neurodegeneration, comprising: (a) a cyclin-E-specific agent; and (b) reagents suitable for detecting cyclin E; wherein the cyclin-E-specific agent is selected from the group consisting of an agent reactive with cyclin E and a nucleic acid probe which hybridizes to nucleic acid encoding cyclin E.

Additional aspects of the present invention will be apparent in view of the description which follows.

### BRIEF DESCRIPTION OF THE FIGURES

FIG. 1 illustrates that parkin interacts specifically with the F-box/WD-repeat protein, hSel-10. (A) Flag-Parkin (52 kDa) was co-expressed with Myc-hSel-10 (69 kDa), Myc-UbcH7 (18 kDa), or PP2A/Bα (55 kDa), in HeLa cells. Anti-Flag immunoprecipitates and lysates were probed, as indicated, by Western blotting. The asterisk indicates the position of an immunoglobulin light chain. (B) Insect cells were co-infected with baculovirus expressing GST-parkin (75 kDa), Flag-hSel-10 (110-kDa form), or Flag-β-TrCP (65 kDa). GST pull-downs, or anti-Flag immunoprecipitations, were performed as described, followed by Western blotting with monoclonal antibodies to either the Flag tag or the parkin ubiquitin homology domain (see FIGS. 9-11). (C) The primary structures of parkin and hSel-10, showing their major domains, are presented. (D) Either wild-type parkin, ARPD mutant (T240R) parkin, a deletion mutant form of parkin lacking the ubiquitin homology domain (ΔUHD parkin), or a truncated form of parkin corresponding to its UHD alone (parkin^{UHD}), was co-expressed in HeLa cells with Myc-tagged hSel-10 (wild-type, mutant WD-repeat alone (hSel-10^{WD}, 49 kDa) or mutant F-box alone (hSel-10^{F-box}, 35 kDa)). Anti-Myc immunoprecipitates and crude lysates were analyzed by Western blotting with polyclonal antibodies to Myc or to the carboxyl terminus of parkin. The parkin polyclonal antibody recognizes both full-length parkin (52 kDa) and a truncated form that is deleted in the UHD (ΔUHD; 42 kDa), and appears to be generated by post-translational processing (Schlossmacher et al., Parkin localizes to the Lewy bodies of Parkinson disease and dementia with Lewy bodies. Am. J. Pathol., 160:1655-67, 2002) (data not shown). (E) Homogenates of 1-g frozen frontal cortex from an ARPD case (see Examples) or an age-matched control were immunoprecipitated with a monoclonal antibody specific for the amino-terminus of human parkin (see FIGS. 9-11), and probed for parkin (using this parkin monoclonal antibody), hSel-10, or α-synuclein. (F) Fresh mouse brain (2 g total) homogenates were incubated with Flag-β-TrCP produced in insect cells or immobilized recombinant Flag-hSel-10 (110 kDa). The 69-kDa and 110-kDa forms of hSel-10 both contain the F-box and WD-repeat domains (Koepp et al., Phosphorylation-dependent ubiquitination of cyclin E by the SCFFbw7 ubiquitin ligase. Science, 294:173-77, 2001). Complexes were Flag-immunoprecipitated and probed by Western blotting for cyclin E (51 kDa) or parkin (using the parkin monoclonal antibody). The asterisk indicates the position of an immunoglobulin heavy chain.

FIG. 2 shows that hSel-10 and UbcH7 function co-operatively to potentiate parkin ubiquitin ligase activity. (A) Plasmids encoding Flag-WT or T240R ARPD parkin were co-transfected, with or without hSel-10 (69-kDa), into HeLa cells. The cells were subsequently treated with lactacystin, to inhibit proteasome function. Cell lysates were immunoprecipitated with an anti-Flag antibody, and probed by Western blotting. (B) Flag-parkin and HA-ubiquitin were co-transfected in HeLa cells, along with full-length hSel-10 (69 kDa), hSel-10^{WD}, hSel-10^{F-box}, β-TrCP, or vector. Lysates were immunoprecipitated with anti-Flag antibody, and ubiquitinated species were detected by Western blotting with an anti-HA antibody. Autoradiography exposure time was extended in (B), relative to the other panels (cf. (B), lane 1; (C), lane 1), to allow for detection of the lower levels of auto-ubiquitination observed in the presence of mutant forms of hSel-10. (C, D) Plasmids encoding Flag-parkin, hSel-10, ubiquitin, UbcH7, or UbcH8, were co-transfected in the combinations indicated, and ubiquitinated species were detected as above.

FIG. 3 illustrates that parkin associates with Cul-1, but not with Skp1 or Rbx1. (A) HeLa cells were transiently transfected with expression constructs encoding Flag-parkin, HA-Cul-1 (86 kDa), and His₆-Skp1(19 kDa), in the presence or absence of Myc-hSel-10 (69 kDa). Lysates were immunoprecipitated with anti-Flag antibodies, and probed by Western blotting, as indicated. (B) Left panel: Insect cells were co-infected with baculoviruses expressing GST-parkin, HA-Cul-1, His₆-Skp1, Rbx1 (11 kDa), and either Flag-hSel-10 (110 kDa) or Flag-β-TrCP. GST-parkin was pulled down with Sepharose-glutathione beads, and complexes were probed by Western blotting. Right panel: Insect cells were infected as above, with or without His₆-Skp1. Skp1-associated complexes were isolated from cell lysates by nickel-agarose pull-downs, and analyzed by Western blotting. (C) Homogenates of 1 g of frozen frontal cortex from an ARPD case or an age-matched control were immunoprecipitated with a monoclonal antibody specific for parkin, and probed, as indicated, by Western blotting. Western-blot analysis of parkin and hSel-10 is shown in FIG. 1E.

FIG. 4 demonstrates that cyclin E is a candidate substrate of the parkin/Cul-1/hSel-10 ubiquitin ligase complex. (A) Insect cells were co-infected with baculoviruses expressing Flag-hSel-10 (110 kDa) or Flag-β-TrCP, GST-parkin, HA-CDK2, and either His₆-cyclin E or His₆-cyclin A1 (50 kDa). Cyclin-E- or cyclin-A1-associated complexes were isolated from cell lysates by nickel-agarose pull-downs, and analyzed by Western blotting, as indicated. (B) Flag-parkin-associated complexes (immunoprecipitated from HeLa cells transfected with Flag-tagged wild-type or T240R mutant parkin) were incubated with recombinant His₆-cyclin E, HA-CDK2, E1, UbcH7, and ubiquitin, in the presence of an ATP-regenerating system. Recombinant His₆-cyclin E generated in insect cells appears as a 51 -kDa band (arrow) and a minor contaminating species at 95-kDa. The asterisk indicates the position of an immunoglobulin heavy chain. (C) Parkin deficiency leads to cyclin E accumulation. Dissociated cortical neurons from E16.5 mice were cultured as described (see Examples), transfected with 25 nM parkin siRNA or control (DAT) siRNA, and then treated for 24 h with 500 μM kainate. After 48 h, cells were extracted with loading buffer, and lysates were probed, by Western blotting, for parkin, cyclin E, β-Actin, and cleaved PARP. The asterisk indicates a non-specific band; the arrow indicates the position of parkin (52 kDa). Densitometric analysis of protein bands (NIH Image 1.62) and relative band intensities are presented as the mean ± SEM of three independent measurements. *=p<0.01, Student's t test (D) Homogenates of substantia nigra (SN) tissue from ARPD brain age-matched control, 2 sporadic PD cases, and 2 sporadic Alzheimer's disease (AD) cases were probed, by Western blotting, for parkin, cyclin E, UbcH7, or cyclin D1 (35 kDa).

FIG. 5 shows that parkin overexpression attenuates the accumulation of cyclin E in kainate-treated cells. Cerebellar granule cells from post-natal day 6 (P6) mice were transfected in suspension with a bicistronic expression plasmid for wild-type parkin (along with GFP) or with vector (GFP alone), cultured at a density of 75,000 cells/cm² for 72 h, and then treated with or without 500 μM kainate for 24 h. (A) Cells were extracted directly with loading buffer, and lysates were analyzed by Western blotting, as indicated. (B-M) Granule cell cultures were fixed, stained with a specific antibody against cyclin E, and then visualized by fluorescence microscopy for cyclin E (red) and GFP (green). Arrows indicate parkin-transfected neurons that display reduced accumulation of cyclin E relative to surrounding untransfected neurons (panels K, K′) or neurons transfected with vector (panels H, H′). scale bar=50 μm

FIG. 6 illustrates that parkin protects post-mitotic neurons from kainate-mediated toxicity. Cerebellar granule cells from P6 mice were transfected as in FIG. 6, and then cultured in the presence or absence of kainate (500 μM) for 24 h. (A-L) Cells were stained for 20 min with 0.5 μg/ml Hoechst dye, and apoptotic nuclei were visualized by fluorescence microscopy. Arrows point to transfected apoptotic neurons apparent in the vector-only transfected cultures (panel K) but not the parkin-transfected culture (panel L). scale bar=100 μm (M) Cell protection in the absence or presence of kainate is expressed as a percentage of GFP-positive (transfected) cells that are also Hoechst-positive (apoptotic). Data are shown as the mean±SEM for 2 independent experiments performed in triplicate. Statistical significance was assessed using one-way ANOVA with Tukey-Krarner post-hoc tests between each group. *=p<0.005

FIG. 7 shows that parkin deficiency potentiates kainate-mediated toxicity in midbrain dopamine neurons. Dissociated midbrain cultures from El 3.5 mice were prepared as described (see Examples), transfected with 25 nM parkin or control (SERT) siRNA, and treated for 24 h with 250 μM kainate (A-X) or 1 μM MPP⁺ (U—X). Cells were treated with Hoechst dye, fixed, and stained with rabbit polyclonal antibodies against either mouse parkin or cyclin E (green) and a rat monoclonal antibody against DAT (red). Immunostaining and apoptotic nuclei were visualized by fluorescence microscopy. Arrows point to examples of DAT-positive, cyclin-E-positive neurons (S) and DAT-positive neurons with apoptotic (Hoechst-positive) nuclei (I, J, and T). Total DAT-specific immunoreactivity (pixels), across 9 fields of view at 20×, was quantified in triplicate using Image software (Scion). Cytoplasmic parkin and cyclin E immunoreactivity (mean pixel density), in DAT-positive neurons, were similarly quantified. Data are shown as the mean±SEM. Statistical significance was assessed using one-way ANOVA with Tukey-Kramer post-hoc tests between each group. *=p<0.01; scale bar=150 μm

FIG. 8 demonstrates that parkin overexpression protects midbrain dopamine neurons from kainate-mediated toxicity. Primary E13.5 midbrain cultures were prepared as above, infected with human parkin or control (GFP) lentiviral vectors (see Examples), and subsequently cultured for 24 h with (G-L) or without (A-F) 250 μM kainate. Cultures were subsequently fixed and stained with a monoclonal antibody specific for human parkin (which is not cross-reactive with the endogenous mouse parkin; see FIGS. 9-12) and a rat monoclonal antibody against DAT. The arrow points to an example of a GFP-infected, kainate-treated, DAT-positive neuron with diminished DAT immunoreactivity. Total DAT-specific immunoreactivity (pixels), across 9 fields of view at 20×, was quantified in triplicate, as in FIG. 7. Data are shown as the mean±SEM. Statistical significance was assessed using one-way ANOVA with Tukey-Kramer post-hoc tests between each group. *=p<0.01; scale bar=150 μm

FIG. 9 sets forth additional Western-blot analyses. (A) Monoclonal antibody 2E10 recognizes the amino-terminal UHD of human parkin. HeLa cells were transfected with wild-type or a UHD-deletion form of parkin, and cell lysates were probed by Western blotting with 2E10 or a polyclonal antibody that recognizes the carboxyl-terminus of parkin (see Examples). A 52-kDa species is recognized by both antibodies; in contrast. the 42-kDa polypeptide appears to represent a processed form of parkin, and is recognized by the polyclonal antibody (Schlossmacher et al., Parkin localizes to the Lewy bodies of Parkinson disease and dementia with Lewy bodies. Am. J. Pathol., 160:1655-67, 2002) (data not shown). (B) Full-length parkin interacts with full-length and deletion forms of hSel-10. HeLa cells were transiently transfected with expression vectors encoding Flag-tagged parkin, or Myc-tagged wild-type or Myc-tagged deletion forms of h-Sel-10. Anti-Flag immunoprecipitates were analyzed by Western blotting, as indicated. (C) The T240R mutation of parkin attenuates the interaction between parkin and Cul-1. HeLa cells were transiently transfected with expression vectors encoding Flag-tagged wild-type or T240R ARPD mutant forms of parkin, along with tagged forms of hSel-10 and Cul-1. Anti-Flag immunoprecipitates were analyzed by Western blotting as indicated. (D) HSel-10 interacts with both parkin and SCF complex components. Insect cells were co-infected with baculoviruses expressing GST-parkin, HA-Cul-1, His₆-Skp1, and Rbx1, with or without Flag-hSel-10 (110 kDa). Anti-Flag immunoprecipitates were analyzed by Western blotting, as indicated.

FIG. 10 further illustrates parkin/cyclin E interaction. (A) Altered parkin expression does not affect cyclin E mRNA levels. Total RNA was extracted from granule cell cultures transfected with parkin or vector alone (see FIG. 5), and from frontal cortex tissue from parkin-deficient ARPD (or age-matched control; see FIG. 4). Cyclin E and β-actin mRNA levels were determined by quantitative RT-PCR, as described (Troy et al., Death in the balance: alternative participation of the caspase-2 and -9 pathways in neuronal death induced by nerve growth factor deprivation. J. Neurosci., 21:5007-16, 2001). (B-D) Both cyclin E immunoreactivity (p<0.05) and apoptosis (p<0.05) are increased in DAT-negative neurons of primary midbrain cultures treated with parkin siRNA (and kainate) relative to control siRNA (and kainate). However, the increased cyclin E immunoreactivity and apoptosis are both less marked than in DAT-positive neurons (p<0.05 for both measures). DAT-negative neurons in midbrain cultures were analyzed as in FIG. 7; DAT-positive neuron data is from FIG. 7. Cytoplasmic parkin and cyclin E immunoreactivity (mean pixel density) in DAT-negative neurons were quantified in triplicate, across 9 fields of view at 20×. Data are shown as the mean±SEM. Statistical significance was assessed using one-way ANOVA with Tukey-Kramer post-hoc tests between each group. *=p<0.01; **=p<0.05; scale bar=150 μm (E-J) Human parkin lentiviral vectors efficiently infect cultured murine midbrain dopamine neurons. E13.5 murine midbrain cultures were infected with lentiviruses encoding human parkin or control (GFP), as described in FIG. 8. Fixed cells were immunostained with the human parkin-specific monoclonal antibody, 2E10 (which does not cross-react with endogenous murine parkin; panels F and I), and a rat antibody against the dopamine transporter (panel G). scale bar=150 μm

FIG. 11 demonstrates that parkin overexpression does not appear to protect dopamine neurons from MPP⁺. (A-F) Murine midbrain cultures were infected with lentiviruses encoding GFP or human parkin, as described in FIG. 8, treated for 24 h with 10 μM MPP⁺, and immunostained with the human parkin antibody (red) and a DAT-specific antibody (green). scale bar=150 μm (G) DAT immunoreactivity was measured and analyzed statistically, as described in FIG. 7.

FIG. 12 sets forth results of analyses using frontal cortex. (A) Frontal cortex extracts from three additional ARPD cases and three additional age-matched controls were prepared as described in FIG. 4D, and analyzed by Western blotting for cyclin E and UbcH7. (B) Cyclin E is variably elevated in extracts of frontal cortex from sporadic AD and PD patients. Frontal cortex extracts from parkin-deficient ARPD cases and age-matched controls (as in A), Huntington's disease (HD) cases, sporadic Parkinson's disease (PD), and sporadic Alzheimer's disease (AD) were prepared as described in FIG. 4D, and analyzed by Western blotting for cyclin E and UbcH7. (C) Most DAT-negative cells in E14 primary midbrain cultures are GABAergic. Embryonic midbrain cultures, as described in FIG. 7, were stained for DAT (red) or GAD-65 (green). scale bar=50 μm

FIG. 13 sets forth the amino acid sequence of parkin protein.

### DETAILED DESCRIPTION OF THE INVENTION

Parkin was initially cloned as a gene that is mutated in rare cases of juvenile-onset Parkinson's Disease. Recent epidemiological studies have indicated that parkin is also defective in a significant percentage of all cases of familial Parkinson's disease. Loss of parkin in familial Parkinson's disease patients leads to decreased survival and/or decreased function of dopaminergic neurons in the midbrain, the pathological hallmark of Parkinson's disease. Accordingly, it is possible that overexpression of parkin might protect these neurons.

Mutations in parkin, which encodes a RING domain protein associated with ubiquitin ligase activity, lead to autosomal recessive Parkinson's disease, which is characterized by midbrain dopamine neuron loss. The inventors have shown that parkin functions in a multiprotein ubiquitin ligase complex that includes the F-box/WD-repeat protein, hSel-10, and Cullin-1. HSel-10 serves to target the parkin ubiquitin ligase activity to cyclin E, a 50-kD, hSel-10-interacting protein previously implicated in the regulation of neuronal apoptosis. Cyclin E complexes with CDK2 in the late G1 phase of the cell cycle, and is known to be upregulated in the context of neuronal excitotoxicity. Consistent with the notion that cyclin E is a substrate of the parkin ubiquitin ligase complex, parkin deficiency potentiates the accumulation of cyclin E in cultured post-mitotic neurons exposed to the glutamatergic excitotoxin, kainate, and promotes their apoptosis. Furthermore, parkin overexpression attenuates the accumulation of cyclin E in toxin-treated primary neurons, including midbrain dopamine neurons, and protects them from apoptosis.

Accordingly, the present invention provides a parkin-associated complex, comprising parkin in association with, or binding with, hSel-10 and cullin-1. In such a complex, amino acid residues of parkin are in direct van der Waa1 and/or hydrogen bond and/or salt-bridge contact with the amino acid residues of hSel-10 and cullin-1.

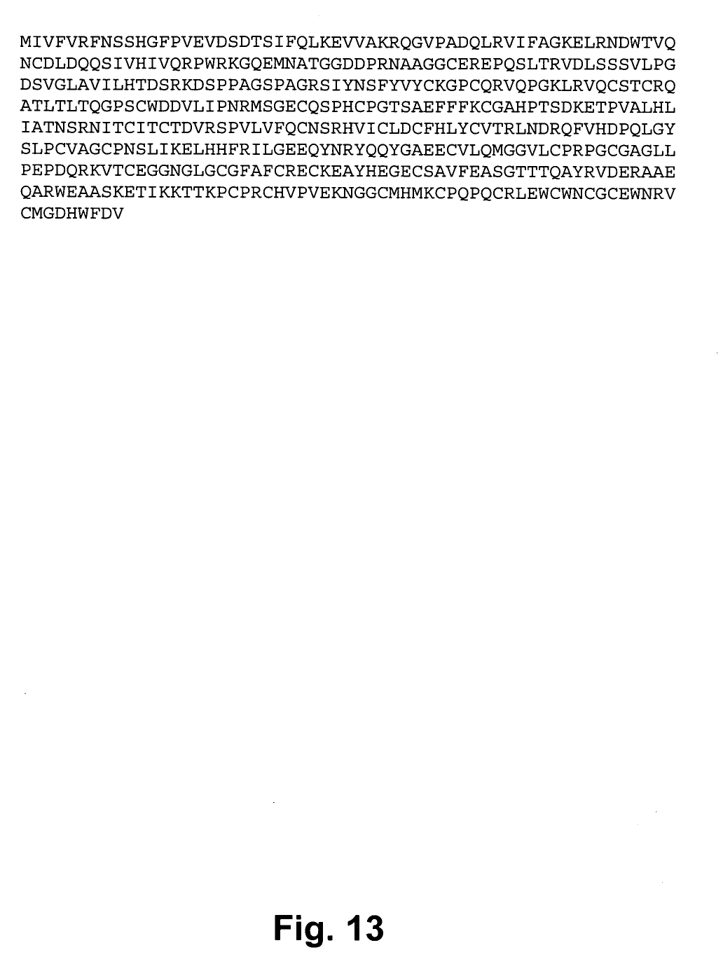

Unless otherwise indicated, “parkin” includes both a “parkin peptide” and a “parkin analogue”. A “parkin peptide” includes at least the carboxyl terminus domain of parkin (including conservative substitutions thereof), from residues 76-465, up to and including a “parkin protein” having the amino acid sequence set forth in FIG. 13 (including conservative substitutions thereof). Unless otherwise indicated, “protein” shall include a protein, protein domain, polypeptide, or peptide. A “parkin analogue” is a functional variant of the parkin peptide, having parkin biological activity, that has 60% or greater (preferably, 70% or greater) amino-acid-sequence homology with the parkin peptide. As further used herein, the term “peptide biological activity” refers to the activity of a protein or peptide that demonstrates an ability to associate physically with, or bind with, hSel-10 (i.e., binding of approximately two fold, or, more preferably, approximately five fold, above the background binding of a negative control), under the conditions of the assays described herein, although affinity may be different from that of parkin.

As used herein, “conservative substitutions” are those amino acid substitutions which are functionally equivalent to a substituted amino acid residue, either because they have similar polarity or steric arrangement, or because they belong to the same class as the substituted residue (e.g., hydrophobic, acidic, or basic). The term “conservative substitutions”, as defined herein, includes substitutions having an inconsequential effect on the ability of parkin to interact with hSel-10 and Cul-1, particularly in respect of the use of said interaction for the identification and design of agonists of the parkin-associated complex, for molecular replacement analyses, and/or for homology modeling.

It will be obvious to the skilled practitioner that the numbering of amino acid residues in parkin, or in the parkin analogues or mimetics covered by the present invention, may be different than that set forth herein, or may contain certain conservative amino acid substitutions that produce the same parkin-hSel-10 associating activity as that described herein. Corresponding amino acids and conservative substitutions in other isoforms or analogues are easily identified by visually inspecting the relevant amino acid sequences, or by using commercially available homology software programs.

In one embodiment of the present invention, the parkin-associated complex comprises the full amino acid sequence of parkin complexed with the full amino acid sequences of hSel-10 and Cul-1. In another embodiment, the complex of the present invention comprises at least the carboxyl terminus domain of parkin, which contains an hSel-10 binding site or association site. As used herein, the “carboxyl terminus domain of parkin” means residues 76-465 of parkin, as well as analogues thereof. Moreover, as used herein, a “binding site” refers to a region of a molecule or molecular complex that, as a result of its shape and charge potential, favorably interacts or associates with another agent—including, without limitation, a protein, polypeptide, peptide, nucleic acid (including DNA or RNA), molecule, compound, antibiotic, or drug—via various covalent and/or non-covalent binding forces.

More specifically, the parkin-associated complex comprises two RING finger domains, including the amino acid residue T240. Accordingly, as contemplated by the present invention, a “hSel-10-binding site of parkin” is a binding site on parkin that, as a result of its shape, reactivity, charge, potential, and other characteristics, favorably interacts or associates with another agent, including, without limitation, a protein (e.g., hSel-10), polypeptide, peptide, nucleic acid (e.g., DNA or RNA), molecule, compound, antibiotic, or drug.

An hSel-10-binding site of parkin may include the actual site on parkin of hSel-10 binding. An hSel-10-binding site also may include accessory binding sites on parkin, adjacent or proximal to the actual site of hSel-10 binding, that nonetheless may affect parkin or hSel-10-parkin activity upon interaction or association with a particular agent—either by direct interference with the actual site of hSel-10 binding, or by indirectly affecting the steric conformation or charge potential of the parkin molecule, and thereby preventing or reducing hSel-10 binding to parkin at the actual site of hSel-10 binding.

Identification of a binding site of a molecule or molecular complex is important because the biological activity of the molecule or molecular complex frequently results from interaction between an agent/ligand and one or more binding sites of the molecule or molecular complex. Therefore, localization of an hSel-10-binding site of parkin provides the most suitable tool for identifying inhibitors that affect the activity of parkin, hSel-10, or Cul-1 or parkin-hSel-10. Localization of a hSel-10-binding site of parkin also permits the use of various molecular design and analysis techniques for the purpose of designing and synthesizing chemical agents capable of favorably associating or interacting with a hSel-10 binding site of parkin or a parkin analogue, wherein said chemical agents potentially act as inhibitors of parkin or parkin-hSel-10 activity. In view of the foregoing, the parkin-hSel-10 interaction and the parkin-hSel-10 complex of the present invention may be used as tools in the rational design and development of drug screens, as a target for small-molecule inhibitors that can act as inhibitor agents or modulators, and as a basis for peptidomimetics.

The parkin-associated complex of the present invention has ubiquitin ligase activity, and can promote ubiquitination of cellular substrates, including cyclin E. Thus, in one embodiment, the parkin-associated complex further comprises cyclin E.

In view of its ubiquitin ligase activity, the parkin-associated complex of the present invention may be useful for promoting ubiquitination of targeted substrates, including cyclin E. Accordingly, the present invention also provides a method for promoting ubiquitination of cyclin E in a post-mitotic neuron. The method of the present invention comprises increasing activity of a parkin-associated complex in the neuron, wherein the parkin-associated complex comprises parkin, hSel-10, and cullin-1.

As used herein, the term “promoting ubiquitination” means augmenting, enhancing, improving, increasing, or inducing the binding of ubiquitin to cyclin E. As further used herein, a “neuron” is a conducting or nerve cell of the nervous system that typically consists of a cell body (perikaryon) that contains the nucleus and surrounding cytoplasm; several short, radiating processes (dendrites); and one long process (the axon), which terminates in twig-like branches (telodendrons), and which may have branches (collaterals) projecting along its course. Additionally, as used herein, the term “post-mitotic” refers to a neuron that is in G0 phase (a quiescent state), and is no longer dividing or cycling.

The post-mitotic neuron of the present invention may be any neuron of the central nervous system (CNS), but is preferably a neuron from the brain. Examples of CNS neurons include, without limitation, cerebellar neurons, or neurons from the cerebellum (e.g., basket cells, Golgi cells, granule cells, Purkinje cells, and stellate cells); cortical neurons, or neurons from the cerebral cortex (e.g., pyramidal cells and stellate cells, including interneurons, midbrain neurons, and neurons of the substantia nigra); hippocampal cells, or cells from the hippocampus (including granule cells); cells of the Pons; and primary neurons (neurons taken directly from the brain, and, in general, placed into a tissue culture dish). Neurons may secrete, or respond to, a variety of neurotransmitters, including, without limitation, acetylcholine, adrenaline, dopamine, endorphins, enkephalins, GABA (gamma aminobutyric acid), glutamate or glutamic acid, noradrenaline, and serotonin. In one embodiment of the present invention, the neuron is a dopamine neuron. Dopamine (3,4-dihydroxyphenylethylamine) is a hormone-like substance with the chemical formula, C₈H₁₁NO₂. It functions in the nervous system as an important neurotransmitter, and is an intermediate in the production of two hormones, epinephrine (adrenaline) and norepinephrine.

The method of the present invention may be used to promote ubiquitination of cyclin E, or add ubiquitin to cyclin E, in vitro, or in vivo in a subject. As used herein, the “subject” is a mammal, including, without limitation, a cow, dog, human, monkey, mouse, pig, or rat. Preferably, the subject is a human. Ubiquitination of cyclin E may be detected by known procedures, including any of the methods, molecular procedures, and assays disclosed herein. The ability of the parkin-associated complex to modulate ubiquitination of cyclin E renders the complex particularly useful for treating neurodegeneration, particularly parkin-associated neurodegeneration, and neurodegeneration associated with glutamate toxicity. Accordingly, in one embodiment of the present invention, the subject is a human with neurodegeneration.

As used herein, “neurodegeneration” means a condition of deterioration of nervous tissue, particularly neurons, wherein the nervous tissue changes to a lower or less functionally active form. It is believed that, by promoting ubiquitination of cyclin E, the parkin-associated complex of the present invention will be useful for the treatment of conditions associated with neurodegeneration. It is further believed that increasing activity of the parkin-associated complex would be an effective therapy, either alone or in combination with other therapeutic agents that are typically used in the treatment of these conditions.

Neurodegeneration may be caused by, or associated with, a variety of factors, including, without limitation, primary neurologic conditions (e.g., neurodegenerative diseases), central nervous system (CNS) and peripheral nervous system (PNS) traumas, and acquired secondary effects of non-neural dysfunction (e.g., neural loss secondary to degenerative, pathologic, or traumatic events, including stroke). Examples of neurodegenerative diseases include, without limitation, Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease. In one embodiment of the present invention, the neurodegeneration is sporadic Parkinson's disease or autosomal recessive early-onset Parkinson's disease. In another embodiment of the present invention, the neurodegeneration is associated with glutamate excitotoxicity.

In accordance with the method of the present invention, activity of the parkin-associated complex in a neuron may be increased by targeting the complex directly. Additionally, activity of the parkin-associated complex in a neuron may be increased indirectly, by targeting an enzyme or other endogenous molecule that regulates or modulates the functions or levels of the complex, or any of its components, in the neuron. Preferably, activity of the parkin-associated complex in the neuron is enhanced by at least 10% in the method of the present invention. More preferably, activity of the parkin-associated complex is enhanced by at least 20%. Activity of the parkin-associated complex is increased in the neuron by an amount effective to promote ubiquitination of cyclin E in the neuron. This amount may be readily determined by the skilled artisan, based upon known procedures, including analysis of titration curves established in vivo, and methods disclosed herein.

By way of example, activity of the parkin-associated complex in a neuron may be increased by directly or indirectly activating, facilitating, or stimulating the ubiquitin ligase function of the parkin-associated complex in the neuron (e.g., by the modulation or regulation of proteins that interact with the complex). The term “activating” , as used herein, means stimulating or inducing the ubiquitin ligase function of the parkin-associated complex in the neuron, particularly the ubiquitination of cyclin E. In the method of the present invention, the parkin-associated complex in a neuron may be activated, for example, by contacting the neuron with a small molecule or protein mimetic that stimulates the complex or that is reactive with (i.e., has affinity for, binds to, or is directed against) one or more components of the complex.

Activity of the parkin-associated complex in a neuron also may be increased by directly or indirectly causing, inducing, or stimulating the upregulation of expression, within the neuron, of one or more components of the complex. Accordingly, in one embodiment of the present invention, activity of the complex is increased in a neuron by contacting the neuron with a modulator of parkin, hSel-10, or Cul-1 expression, in an amount effective to promote ubiquitination of cyclin E in the neuron. The modulator may be a protein, polypeptide, peptide, nucleic acid (including DNA or RNA), antibody, Fab fragment, F(ab′)₂ fragment, molecule, compound, antibiotic, drug, or an agent reactive with parkin, hSel-10, or Cul-1 that induces or upregulates parkin, hSel-10, or Cul-1 expression.

Modulators of parkin, hSel-10, or Cul-1 maybe identified using a simple screening assay. For example, to screen for candidate modulators of parkin, hSel-10, or Cul-1, midbrain dopamine neurons from an ARPD patient may be plated onto microtiter plates, then contacted with a library of drugs. Any resulting increase in, or upregulation of, parkin, hSel-10, or Cul-1 expression then may be detected using nucleic acid hybridization and/or immunological techniques known in the art, including an ELISA. Additional modulators of parkin, hSel-10, or Cul-1 expression may be identified using screening procedures well known in the art or disclosed herein. Modulators of parkin, hSel-10, or Cul-1 will be those drugs which induce or upregulate expression of parkin, hSel-10, or Cul-1. In this manner, candidate modulators also may be screened for their ability to protect neurons from excitotoxicity, and, therefore, their ability to treat neurodegeneration.

Activity of the parkin-associated complex in a neuron also may be increased in by directly or indirectly increasing levels of the complex, or any of its components, within the neuron. By way of example, the level of parkin, hSel-10, or Cul-1 in a neuron may be increased by contacting the neuron with parkin, hSel-10, or Cul-1 protein, in an amount effective to promote ubiquitination of cyclin E in the neuron. Similarly, the level of parkin, hSel-10, or Cul-1 in a subject may be increased by contacting the neuron with a nucleic acid sequence encoding parkin, hSel-10, or Cul-1, in a manner permitting expression of parkin, hSel-10, or Cul-1 protein in the neuron, and in an amount effective to promote ubiquitination of cyclin E.

In the method of the present invention, ubiquitination of cyclin E may be promoted in the neuron in vitro, or in vivo in a subject, by introducing a parkin-associated protein agent, or a nucleic acid sequence encoding the parkin-associated protein agent, to the neuron, in a manner permitting expression of the protein. The neuron may be contained in nervous tissue of the subject, and may be detected in nervous tissue of the subject by standard detection methods readily determined from the known art, examples of which include, without limitation, immunological techniques (e.g., immunohistochemical staining), fluorescence imaging techniques, and microscopic techniques. In one embodiment of the present invention, ubiquitination of cyclin E is effected in vivo in a subject by administering a protein agent, such as parkin, to the subject.

The present invention contemplates the use of proteins and protein analogues generated by synthesis of polypeptides in vitro, e.g., by chemical means or in vitro translation of mRNA. For example, parkin, hSel-10, or Cul-1 may be synthesized by methods commonly known to one skilled in the art (Modern Techniques of Peptide and Amino Acid Analysis (New York: John Wiley & Sons, 1981; Bodansky, M., Principles of Peptide Synthesis (New York: Springer-Verlag New York, Inc., 1984). Examples of methods that may be employed in the synthesis of the amino acid sequences, and analogues of these sequences, include, but are not limited to, solid-phase peptide synthesis, solution-method peptide synthesis, and synthesis using any of the commercially-available peptide synthesizers. The amino acid sequences of the present invention may contain coupling agents and protecting groups, which are used in the synthesis of protein sequences, and which are well known to one of skill in the art.

In the method of the present invention, a modulator of parkin, hSel-10, or Cul-1 expression, a parkin, hSel-10, or Cul-1 protein, or a nucleic acid sequence encoding parkin, hSel-10, or Cul-1 is administered to a subject who has neurodegeneration in an amount effective to treat the neurodegeneration in the subject. As used herein, the phrase “effective to treat the neurodegeneration” means effective to ameliorate or minimize the clinical impairment or symptoms resulting from the neurodegeneration. For example, where the neurodegeneration is Parkinson's disease, the clinical impairment or symptoms of the neurodegeneration may be ameliorated or minimized by diminishing any pain or discomfort suffered by the subject; by extending the survival of the subject beyond that which would otherwise be expected in the absence of such treatment; by inhibiting or preventing the development or spread of the neurodegeneration, including loss, in the substantia nigra, of nerve cells containing dopamine; and/or by limiting, suspending, terminating, or otherwise controlling tremors, speech impediments, movement difficulties, dementia, and other symptoms associated with Parkinson's disease. The amount of modulator of parkin, hSel-10, or Cul-1 expression, the amount of parkin, hSel-10, or Cul-1 protein, or the amount of nucleic acid encoding parkin, hSel-10, or Cul-1 that is effective to treat neurodegeneration in a subject will vary depending on the particular factors of each case, including the type of neurodegeneration, the stage of neurodegeneration, the subject's weight, the severity of the subject's condition, and the method of administration. These amounts can be readily determined by the skilled artisan.

In the method of the present invention, the modulator of parkin, hSel-10, or Cul-1 expression, the parkin, hSel-10, or Cul-1 protein, or the nucleic acid sequence encoding parkin, hSel-10, or Cul-1 may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous, or subcutaneous administration), transdermal administration, and administration by osmotic pump. One preferred method of administration is parenteral administration, by intravenous or subcutaneous injection.

For oral administration, the formulation of the parkin, hSel-10, or Cul-1 modulator, protein, or nucleic acid may be presented as capsules, tablets, powders, granules, or as a suspension. The formulation may have conventional additives, such as lactose, mannitol, cornstarch, or potato starch. The formulation also may be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the parkin, hSel-10, or Cul-1 modulator, protein, or nucleic acid may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above.

For transdermal administration, the parkin, hSel-10, or Cul-1 modulator, protein, or nucleic acid may be combined with skin penetration enhancers, such as propylene glycol, polyethylene glycol, isopropanol, ethanol, oleic acid, N-methylpyrrolidone, and the like, which increase the permeability of the skin to the modulator, protein, or nucleic acid, and permit the modulator, protein or nucleic acid to penetrate through the skin and into the bloodstream. The composition of enhancer and modulator, protein, or nucleic acid also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The modulator, protein, or nucleic acid may be administered transdermally, at or near the site on the subject where the neoplasm is localized. Alternatively, the modulator, protein, or nucleic acid may be administered transdermally at a site other than the affected area, in order to achieve systemic administration.

The parkin, hSel-10, or Cul-1 modulator, protein, or nucleic acid of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the modulator, protein, or nucleic acid.

In accordance with the methods of the present invention, where the modulator of parkin, hSel-10, or Cul-1 expression is a protein, or where parkin, hSel-10, or Cul-1 protein is the therapeutic of choice, the protein may be administered to a subject by introducing to the subject the protein itself, or by introducing to the subject a nucleic acid encoding the protein in a manner permitting expression of the protein. Accordingly, in one embodiment of the present invention, activity of the parkin-associated complex in one or more neurons in a subject may be increased by administering to the subject an amount of a protein (e.g., a modulator of parkin, hSel-10, or Cul-1 expression, or the parkin, hSel-10, or Cul-1 protein itself). In a further embodiment of the present invention, activity of the parkin-associated complex in one or more neurons in the subject may be increased by administering to the subject a nucleic acid sequence encoding a protein (e.g., a modulator of parkin, hSel-10, or Cul-1 expression, or the parkin, hSel-10, or Cul-1 protein itself), in a manner permitting expression of parkin, hSel-10, or Cul-1 in neurons of the subject.

The proteins of the present invention may be administered or introduced to a subject by known techniques used for the introduction of proteins and other drugs, including, for example, injection and transfusion. Where the neurodegeneration is localized to a particular portion of the body of the subject, it may be desirable to introduce the therapeutic protein directly to that area by injection or by some other means (e.g., by introducing the protein into the blood or another body fluid). The amount of protein to be used is an amount effective to treat neurodegeneration in the subject, as defined above, and may be readily determined by the skilled artisan.

In the method of the present invention, where the modulator of parkin, hSel-10, or Cul-1 expression is a protein, or where parkin, hSel-10, or Cul-1 protein is the therapeutic of choice, the protein also may be administered or introduced to the subject by introducing into a sufficient number of neurons of the subject a nucleic acid encoding the protein, in a manner permitting expression of the protein. The amount of nucleic acid encoding the therapeutic protein is an amount that will produce the protein in an amount effective to treat neurodegeneration, as defined above, in the subject. This amount may be readily determined by the skilled artisan.

Nucleic acid encoding the modulator of parkin, hSel-10, or Cul-1 expression, or the parkin, hSel-10, or Cul-1 protein itself, as well as any nucleotide modulators of parkin, hSel-10, or Cul-1 expression, all may be introduced to the subject using conventional procedures known in the art, including, without limitation, electroporation, DEAE Dextran transfection, calcium phosphate transfection, lipofection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, lentivirus, cytomegalovirus, and vaccinia virus.

It is within the confines of the present invention that a nucleic acid encoding a modulator of parkin, hSel-10, or Cul-1 expression, or the parkin, hSel-10, or Cul-1 protein itself, may be introduced into suitable neurons in vitro, using conventional procedures, to achieve expression of the therapeutic protein in the neurons. Neurons expressing the modulator of parkin, hSel-10, or Cul-1 expression, or the parkin, hSel-10, or Cul-1 protein itself, then may be introduced into a subject to treat neurodegeneration in vivo. In such an ex vivo gene therapy approach, the neurons are preferably removed from the subject, subjected to DNA techniques to incorporate nucleic acid encoding the therapeutic protein, and then reintroduced into the subject.

It is also within the confines of the present invention that a formulation containing a parkin, hSel-10, or Cul-1 modulator, protein, or nucleic acid may be further associated with a pharmaceutically-acceptable carrier, thereby comprising a pharmaceutical composition. Accordingly, the present invention further provides a pharmaceutical composition, comprising a modulator of parkin, hSel-10, or Cul-1 expression, or a parkin, hSel-10, or Cul-1 protein or a nucleic acid sequence encoding parkin, hSel-10, or Cul-1, and a pharmaceutically acceptable carrier. The pharmaceutically acceptable carrier must be “acceptable” in the sense of being compatible with the other ingredients of the composition, and not deleterious to the recipient thereof. The pharmaceutically acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical composition of the present invention may be prepared by methods well-known in the pharmaceutical arts. For example, the composition may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the vaccine. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered orally, topically, or by injection, including, but not limited to, intravenous, intraperitoneal, subcutaneous, and intramuscular injection.

In one embodiment of the present invention, the pharmaceutical composition is a therapeutic composition comprising a nucleic acid encoding a parkin-associated agent (e.g., a parkin protein, a parkin mimetic, a modulator of parkin expression, and a modulator of parkin activity), a lentiviral vector, and, optionally, a pharmaceutically-acceptable carrier. By way of example, a parkin lentiviral vector may be assembled by cloning the human parkin cDNA into the BamH1 and XhoI restriction enzyme sites of plasmid pTRIP GFP, and replacing the GFP gene (Zennou et al., The HIV-1 DNA flap stimulates HIV vector-mediated cell transduction in the brain. Nat. Biotechnol., 19:446-50, 2001). A parkin virus may be produced by co-transfection of 293T cells with p8.91 and pHCMV-G, and viral transduction of neuronal cultures may be performed as described (Zennou et al., The HIV-1 DNA flap stimulates HIV vector-mediated cell transduction in the brain. Nat. Biotechnol., 19:446-50, 2001).

The formulations of the present invention may be prepared by methods well-known in the pharmaceutical arts. For example, the parkin, hSel-10, or Cul-1 modulator, protein, or nucleic acid may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the parkin, hSel-10, or Cul-1 modulator, protein, or nucleic acid of the present invention to a subject to treat neurodegeneration. The parkin, hSel-10, or Cul-1 modulator, protein, or nucleic acid is provided in an amount that is effective to treat neurodegeneration in a subject to whom the pharmaceutical composition is administered. That amount may be readily determined by the skilled artisan, as described above.

It is within the confines of the present invention that the modulator of parkin, hSel-10, or Cul-1 expression may be linked to another agent, or administered in combination with another agent, such as a drug or a ribozyme, in order to increase the effectiveness of the treatment of neurodegeneration, increase the efficacy of targeting, and/or increase the efficacy of cyclin E ubiquitination. Furthermore, in accordance with the method of the present invention, parkin, hSel-10, or Cul-1 protein may be administered to a subject who has neurodegeneration, either alone or in combination with one or more drugs used to treat neurodegeneration, including Parkinson's disease. Examples of drugs used to treat Parkinson's disease include, without limitation, deprenyl, selenium, and vitamin E.

The pharmaceutical composition of the present invention may be useful for treating neurodegeneration in a subject. Accordingly, the present invention further provides a method for treating neurodegeneration in a subject in need of treatment, comprising administering to the subject a pharmaceutical composition comprising a parkin, hSel-10, or Cul-1 modulator, protein, or nucleic acid, and a pharmaceutically-acceptable carrier. The parkin, hSel-10, or Cul-1 modulator, protein, or nucleic acid is provided in an amount that is effective to treat the neurodegeneration in a subject to whom the composition is administered. This amount may be readily determined by the skilled artisan. In one embodiment of the present invention, the pharmaceutical composition comprises a nucleic acid encoding a parkin-associated agent (e.g., a parkin protein, a parkin mimetic, a modulator of parkin expression, and a modulator of parkin activity), a lentiviral vector, and, optionally, a pharmaceutically-acceptable carrier. In a preferred embodiment of the present invention, the neurodegeneration is sporadic Parkinson's disease or autosomal recessive early-onset Parkinson's disease.

The pharmaceutical composition of the present invention may also be useful for studying treatment options in animal models of neurodegeneration, including Parkinson's disease. In particular, because lentivirus vectors are potentially useful, in vivo, for gene therapy, the present invention may provide an animal model demonstrating the efficacy of using parkin-encoding lentivirus in Parkinson's disease. Accordingly, the present invention also provides for use of the pharmaceutical composition of the present in an animal model of neurodegeneration (e.g., Parkinson's disease). In one embodiment of the present invention, the pharmaceutical composition comprises a nucleic acid encoding a parkin-associated agent (e.g., a parkin protein, a parkin mimetic, a modulator of parkin expression, and a modulator of parkin activity), a lentiviral vector, and, optionally, a pharmaceutically-acceptable carrier. In a preferred embodiment of the present invention, the neurodegeneration is sporadic Parkinson's disease or autosomal recessive early-onset Parkinson's disease.

The present invention also provides a method for identifying an agent that interacts with a parkin-associated complex, by assessing the ability of a candidate agent to enhance interaction between the complex and cyclin E, wherein the parkin-associated complex comprises parkin, hSel-10, and cullin-1. The method of the present invention comprises the steps of: (a) contacting a candidate agent with the complex, in the presence of cyclin E; and (b) assessing the ability of the candidate agent to enhance interaction between the complex and cyclin E. As used herein, an “agent” shall include a protein, polypeptide, peptide, nucleic acid (including DNA or RNA), antibody, Fab fragment, F(ab′)₂ fragment, molecule, antibiotic, drug, compound, and any combination thereof. A Fab fragment is a univalent antigen-binding fragment of an antibody, which is produced by papain digestion. An F(ab′)₂ fragment is a divalent antigen-binding fragment of an antibody, which is produced by pepsin digestion.

An agent that enhances interaction between the parkin-associated complex and cyclin E may be either natural or synthetic, and may be an agent reactive with parkin, hSel-10, or cullin-1 (i.e., has affinity for, binds to, or is directed against parkin, hSel-10, or cullin-1). An agent that is reactive with parkin, hSel-10, and cullin-1, as disclosed herein, may have the ability to enhance interaction between parkin, hSel-10, or Cul-1 and cyclin E by binding to parkin, hSel-10, or Cul-1. A candidate agent having the ability to bind to parkin, hSel-10, or Cul-1 would, as a consequence of this binding, either enhance activity of the parkin-associated complex through steric interactions, or mimic parkin, hSel-10, or Cul-1 in their ubiquitin ligase activity, thereby reinforcing the ubiquitinating power of parkin, hSel-10, Cul-1, or the parkin-associated complex.

According to the method of the present invention, an agent that enhances interaction between the parkin-associated complex and cyclin E may be identified using an in vitro assay (e.g., a direct binding assay, an assay of ubiquitin ligase activity, etc.). For instance, in a direct binding assay, the binding of a candidate agent to the parkin-associated complex, including any of its constituent components, may be measured directly. The candidate agent may be supplied by a peptide library, for example.

The assay formats of the present invention employ labeled assay components. Labeling of parkin, hSel-10, or Cul-1 may be accomplished using one of a variety of different chemiluminescent and radioactive labels known in the art, including any of those described above. Qualitative results then may be obtained by competitive autoradiographic-plate binding assays; alternatively, Scatchard plots may be used to generate quantitative results. The labels of the present invention may be coupled directly or indirectly to the desired component of the assay, according to methods well known in the art. The choice of label depends on a number of relevant factors, including the sensitivity required, the ease of conjugation with the compound to be labeled, stability requirements, and available instrumentation.

Under specified assay conditions, a candidate agent is considered to be capable of enhancing interaction between the parkin-associated complex and cyclin E if the interaction is increased by 50% (preferably 90%) or more. Where a direct binding assay configuration is used, a candidate agent is considered to bind the parkin-associated complex when the signal measured is twice the background level or higher.

As disclosed herein, cyclin E has been implicated in a number of biological events in post-mitotic neurons. For example, it has been shown that accumulation of cyclin E may be associated with Parkinson's disease and other neurodegenerative disorders, and that this accumulation may be the result of diminished ubiquitination of cyclin E. It has also been demonstrated that accumulation of cyclin E in post-mitotic neurons may lead to apoptosis, and that this apoptotic death may be correlated with Parkinson's disease.

In view of the foregoing, it is clear that therapeutics designed around, or similar to, the structure of parkin, hSel-10, or Cul-1 may be useful in treating a number of cyclin-E-associated biological events in post-mitotic neurons, including accumulation of cyclin E, diminished ubiquitination of cyclin E, and cyclin-E-associated apoptosis. Thus, once the candidate agent of the present invention has been screened, and has been determined to have suitable binding affinity to the parkin-associated complex (e.g., it is reactive with the complex), or to enhance interaction between the complex and cyclin E, it may be evaluated to ascertain whether it has an effect on biological events or processes in which cyclin E has been implicated, including accumulation of cyclin E, diminished ubiquitination of cyclin E, cyclin-E-associated apoptosis, and cyclin-E-associated neurodegeneration. It is expected that the candidate agent of the present invention will be useful to treat neurodegenerations, including those disclosed herein.

Accordingly, the present invention further comprises the steps of: (c) contacting the candidate agent with one or more post-mitotic neurons containing cyclin E; and (d) determining if the agent has an effect on a cyclin-E-associated biological event in the one or more neurons. As used herein, a “cyclin-E-associated biological event” includes a biochemical or physiological process in which cyclin E levels or activity have been implicated. As disclosed herein, examples of cyclin-E-associated biological events include, without limitation, accumulation of cyclin E, diminished ubiquitination of cyclin E, cyclin-E-associated apoptosis, and cyclin-E-associated neurodegeneration. As further used herein, a neuron “containing cyclin E” is a neuron in which cyclin E, or a derivative or homologue thereof, is naturally expressed or naturally occurs. According to this method of the present invention, a candidate agent may be contacted with one or more post-mitotic neurons in vitro. For example, a culture of post-mitotic neurons may be incubated with a preparation containing the candidate agent. The candidate agent's effect on a cyclin-E-associated biological event then may be assessed by any biological assays or methods known in the art, including histological analyses.

The present invention is further directed to agents identified by the above-described identification methods. Such agents may be useful for treating a cyclin-E-associated condition. As used herein, a “cyclin-E-associated condition” is a condition, disease, or disorder in which cyclin E levels or activity have been implicated, and includes the following: a cyclin-E-associated biological event, including a cyclin-E-associated neurodegeneration in a subject in need of treatment, and excitotoxicity in post-mitotic neurons. The cyclin-E-associated condition may be treated in the subject by administering to the subject an amount of the agent effective to treat the cyclin-E-associated condition in the subject. This amount may be readily determined by one skilled in the art.

Accordingly, in one embodiment, the present invention provides a method for protecting one or more post-mitotic neurons from excitotoxicity, by contacting the neuron(s) with the above-described agent, in an amount effective to protect the neuron(s) from excitotoxicity. Excitotoxicity is a characteristic of a substance that is capable of exciting, and then poisoning, cells or tissues. An example of excitotoxicity is nerve injury produced by glutamate. Glutamatergic excitotoxicity has previously been implicated in Parkinson's disease and in other neurodegenerative disorders, stroke, and traumatic brain injury. In a preferred embodiment of the present invention, the neuron is a dopamine neuron.

Because glutamatergic excitotoxicity has previously been implicated in Parkinson's disease and in other neurodegenerative disorders, stroke, and traumatic brain injury, the present invention may be useful as a method of protecting dopaminergic neurons in familial and sporadic Parkinson's disease; other common neurological diseases, such as Alzheimer's disease; and stroke. Accordingly, in another embodiment, the present invention provides a method for treating or preventing neurodegeneration in a subject, by administering to the subject the above-described agent, in an amount effective to treat or prevent the neurodegeneration in the subject. In a preferred embodiment of the present invention, the neurodegeneration is selected from the group consisting of Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease. Preferably, the neurodegeneration is sporadic Parkinson's disease or autosomal recessive early-onset Parkinson's disease, or is associated with glutamate excitotoxicity.

The present invention also provides a pharmaceutical composition comprising the agent identified by the above-described identification method and a pharmaceutically-acceptable carrier. Examples of suitable pharmaceutically-acceptable carriers, and methods of preparing pharmaceutical formulations and compositions, are described above. The pharmaceutical composition of the present invention would be useful for administering to a subject an agent that is enhances interaction between a parkin-associated complex and cyclin E, in order to treat a cyclin-E-associated condition, wherein the complex comprises parkin, hSel-10, and Cul-1. In such cases, the pharmaceutical composition is administered to a subject in an amount effective to treat the cyclin-E-associated condition.

The present invention further provides a method for decreasing cyclin E in one or more post-mitotic neurons, comprising contacting the neuron(s) with a parkin-associated agent. The parkin-associated agent may be a parkin protein, a parkin mimetic, a modulator of parkin expression, and a modulator of parkin activity. The parkin-associated agent is used in an amount effective to decrease cyclin E in the neuron(s). This amount may be readily determined by the skilled artisan, based upon known procedures, including analysis of titration curves established in vivo, and methods disclosed herein. Cyclin E may be decreased in the neuron(s) by decreasing the level of cyclin E in the neuron(s) or by decreasing accumulation of cyclin E in the neuron(s). As disclosed above, the level of cyclin E in the neuron(s) or accumulation of cyclin E in the neuron(s) may be decreased by promoting ubiquitination of cyclin E in the neuron(s). Levels of cyclin E in the neuron(s) may be measured or detected by known procedures, including Western blotting and any of the methods, molecular procedures, and assays disclosed herein.

The post-mitotic neuron(s) of the present invention may be any of those described above. In one embodiment, the neuron is a cerebellar granule neuron, a cortical neuron, or a substantia nigra neuron. As discussed herein, accumulation of cyclin E in post-mitotic neurons has been associated with damage resulting from excitotoxicity. Accordingly, in another embodiment of the present invention, the neuron is damaged. Preferably, the damage is associated with excitotoxicity; more preferably, the damage is associated with glutamate excitotoxicity.

The method of the present invention may be used to decrease cyclin E in post-mitotic neurons in vitro, ex vivo, or in vivo in a subject, in accordance with methods described above. The neuron(s) may be contacted with the parkin-associated agent in vivo in a subject by administering the parkin-associated agent to the subject. The subject may be any of those described above, but is preferably a human. In one embodiment of the present invention, the subject has neurodegeneration.

It is believed that, by decreasing cyclin E in post-mitotic neurons, parkin-associated agents will be useful for the treatment of conditions associated with accumulation of cyclin E in post-mitotic neurons, including Parkinson's disease and other neurodegenerative diseases. It is further believed that the parkin-associated agents would be effective either alone or in combination with other therapeutic agents that are typically used in the treatment of these conditions. Accordingly, the present invention provides a method for treating neurodegeneration in a subject in need of treatment, comprising contacting at least one post-mitotic neuron in the subject with an amount of a parkin-associated agent effective to decrease cyclin E in the neuron, thereby treating the neurodegeneration. Examples of neurodegeneration, including Parkinson's disease, which may be treated by the method of the present invention are discussed above. In a preferred embodiment of the present invention, the neurodegeneration is sporadic Parkinson's disease or autosomal recessive early-onset Parkinson's disease, or is associated with glutamate excitotoxicity.

The inventors have shown that cerebellar granule neurons that are transfected with a parkin construct, such that they over express parkin, are protected from kainate-mediated (glutamatergic) excitotoxicity and apoptotic death. The inventors have also shown that primary dopamine neuron cultures infected with a lentivirus (a genus of the family retroviridae, consisting of non-oncogenic retroviruses that produce multi-organ diseases) that leads to parkin overexpression are protected from glutamatergic excitotoxicity and apoptotic death.

Accordingly, the present invention also provides a method for protecting one or more post-mitotic neurons from the negative or deleterious effects of toxic insults, including excitotoxicity, by contacting the neuron(s) with a parkin-associated agent. The parkin-associated agent may be a parkin protein, a parkin mimetic, a modulator of parkin expression, and a modulator of parkin activity. The parkin-associated agent is used in an amount effective to protect the neuron(s) from toxicity. This amount may be readily determined by the skilled artisan, based upon known procedures, including analysis of titration curves established in vivo, and methods disclosed herein. By protecting the neuron(s) from toxic insults, the parkin-associated agent will enhance the survival of the neuron(s). As used herein, the term “enhance the survival of neurons” means the full or partial protection of neurons from damage, death, degeneration, demyelination, or injury. The survival, and enhancement of the survival, of neurons may be measured or detected by known procedures, including Western blotting for myelin-specific and axon-specific proteins, electron microscopy in conjunction with morphometry, and any of the methods, molecular procedures, and assays disclosed herein.

The post-mitotic neuron(s) of the present invention may be any of those described above. In one embodiment, the neuron is a cerebellar granule neuron, a cortical neuron, or a substantia nigra neuron. In another embodiment, the neuron is a dopamine neuron. The excitotoxicity is preferably glutamate excitotoxicity. More preferably, the excitotoxicity is kainate-mediated glutamate excitotoxicity. The protection of the neuron(s) from excitotoxicity may result in protection of the neuron(s) from apoptosis, as it has been shown that excitotoxicity, particularly glutamate excitotoxicity, leads to apoptotic death of post-mitotic neurons.

The method of the present invention may be used to protect post-mitotic neurons from excitotoxicity in vitro, ex vivo, or in vivo in a subject, in accordance with methods described above. The neuron(s) may be contacted with the parkin-associated agent in vivo in a subject by administering the parkin-associated agent to the subject. The subject may be any of those described above, but is preferably a human. In one embodiment of the present invention, the subject has neurodegeneration.

It is believed that, by protecting post-mitotic neurons from the negative or deleterious effects of toxic insults, parkin-associated agents will be useful for the treatment of Parkinson's disease and other conditions associated with toxicity, particularly excitotoxicity, in post-mitotic neurons. It is further believed that parkin-associated agents would be effective either alone or in combination with other therapeutic agents that are typically used in the treatment of these conditions. Accordingly, the present invention provides a method for treating neurodegeneration in a subject in need of treatment, comprising contacting at least one post-mitotic neuron in the subject with an amount of a parkin-associated agent effective to protect the neuron(s) from the negative or deleterious effects of toxic insults, including excitotoxicity, thereby treating the neurodegeneration. Examples of neurodegeneration, including Parkinson's disease, which may be treated by the method of the present invention are discussed above. In a preferred embodiment of the present invention, the neurodegeneration is sporadic Parkinson's disease or autosomal recessive early-onset Parkinson's disease, or is associated with glutamate excitotoxicity.

In view of the foregoing, the present invention also provides for use of a parkin-associated agent to protect a post-mitotic neuron from excitotoxicity, wherein the neuron is contacted with an amount of parkin-associated agent effective to protect the neuron from excitotoxicity. The parkin-associated agent may be a parkin protein, a parkin mimetic, a modulator of parkin expression, or a modulator of parkin activity. The excitotoxicity is preferably glutamate excitotoxicity. More preferably, the excitotoxicity is kainate-mediated glutamate excitotoxicity.

The inventors have demonstrated herein that cyclin E specifically accumulates in Parkinson's disease, and that diminished parkin levels are partly responsible for this accumulation. Therefore, cyclin E represents a novel marker for Parkinson's disease. Accordingly, the present invention further provides a method for determining whether a subject has neurodegeneration, thereby permitting the diagnosis of such neurodegeneration in the subject. The subject may be any of those described above. Preferably, the subject is a human. Additionally, examples of neurodegeneration have been previously discussed. In one embodiment of the present invention, the neurodegeneration is sporadic Parkinson's disease or autosomal recessive early-onset Parkinson's disease.

The method of the present invention comprises assaying a diagnostic sample of the subject for cyclin E, wherein detection of a cyclin E level elevated above normal is diagnostic of neurodegeneration in the subject. As used herein, “cyclin E” includes both a cyclin E protein and a cyclin E analogue. As further used herein, a “cyclin E analogue” is a functional variant of the cyclin E protein, having cyclin E biological activity, that has 60% or greater (preferably, 70% or greater) amino-acid-sequence homology with the cyclin E protein. A cyclin E “analogue” includes a variant of the cyclin E protein.

In accordance with the method of the present invention, the diagnostic sample of a subject may be assayed in vitro or in vivo. Where the assay is performed in vitro, a diagnostic sample from the subject may be removed using standard procedures. The diagnostic sample may be any nervous tissue, including brain tissue, which may be removed by standard biopsy. In addition, the diagnostic sample may be any tissue known to have neurodegeneration, any tissue suspected of having neurodegeneration, or any tissue believed not to have neurodegeneration. In a preferred embodiment of the present invention, the diagnostic sample is taken from a sample from the frontal cortex, midbrain, or substantia nigra of the subject.

Protein may be isolated and purified from the diagnostic sample of the present invention using standard methods known in the art, including, without limitation, extraction from a tissue (e.g., with a detergent that solubilizes the protein) where necessary, followed by affinity purification on a column, chromatography (e.g., FTLC and HPLC), immunoprecipitation (with an antibody to cyclin E), and precipitation (e.g., with isopropanol and a reagent such as Trizol). Isolation and purification of the protein may be followed by electrophoresis (e.g., on an SDS-polyacrylamide gel). Nucleic acid may be isolated from a diagnostic sample using standard techniques known to one of skill in the art.

In accordance with the method of the present invention, neurodegeneration in a subject is diagnosed by assaying a diagnostic sample of the subject for cyclin E. The level of cyclin E in the sample may be detected by measuring cyclin E amounts in the sample. A diagnostic sample may be assayed for the level of cyclin E by assaying for cyclin E protein, cyclin E cDNA, or cyclin E MRNA. The appropriate form of cyclin E will be apparent based on the particular techniques discussed herein. Preferably, the diagnostic sample of the present invention is assayed for the level of cyclin E protein. It is contemplated that the diagnostic sample may be assayed for expression of any or all forms of cyclin E protein (including precursor, endoproteolytically-processed forms, and other forms resulting from post-translational modification) in order to determine whether a subject or patient has neurodegeneration.

Alternatively, the level of cyclin E in the sample may be detected by detecting below-normal interaction of cyclin E and a parkin-associated complex, wherein the parkin-associated complex comprises parkin, hSel-10, and Cul-1. Accordingly, in one embodiment of the present invention, the level of cyclin E elevated above normal is detected by detecting below-normal interaction of cyclin E and the parkin-associated complex. Methods for detecting interaction between the parkin-associated complex and cyclin E have been discussed above.

As used herein, the term “elevated above normal” means that cyclin E is detected at a level that is significantly greater than the level expected for the same type of diagnostic sample taken from a nondiseased subject or patient (i.e., one who does not have neurodegeneration) of the same gender and of similar age. As further used herein, “significantly greater” means that the difference between the level of cyclin E that is elevated above normal, and the expected (normal) level of cyclin E, is of statistical significance. Preferably, the level of cyclin E elevated above normal is a level that is at least 10% greater than the level of cyclin E otherwise expected in the diagnostic sample. Where cyclin E is expected to be absent from a particular diagnostic sample taken from a particular subject or patient, the normal level of cyclin E for that subject or patient is nil. Where a particular diagnostic sample taken from a particular subject or patient is expected to have a low, constitutive level of cyclin E, that low level is the normal level of cyclin E for that subject or patient. As disclosed herein, cyclin E is generally present at lower levels in post-mitotic neurons that have not been exposed to toxic insults, including excitotoxicity.

Expected or normal levels of cyclin E for a particular diagnostic sample taken from a subject or patient may be easily determined by assaying nondiseased subjects of a similar age and of the same gender. For example, diagnostic samples may be obtained from at least 30 normal, healthy men between the ages of 25 and 80, to determine the normal quantity of cyclin E in males. A similar procedure may be followed to determine the normal quantity of cyclin En in females. Once the necessary or desired samples have been obtained, the normal quantity of cyclin E in men and women may be determined using a standard assay for quantification, such as flow cytometry, Western blot analysis, or an ELISA for measuring protein quantities, as described below. For example, an ELISA may be run on each sample in duplicate, and the mean and standard deviation of the quantity of cyclin E may be determined. If necessary, additional subjects may be recruited before the normal quantity of cyclin E is determined. A similar type of procedure may be used to determine the expected or normal level of interaction between cyclin E and a parkin-associated complex for a particular diagnostic sample taken from a subject or patient.

In accordance with the method of the present invention, a diagnostic sample of a subject may be assayed for cyclin E (or for interaction between cyclin E and a parkin-associated complex), and cyclin E (or interaction between cyclin E and a parkin-associated complex) may be detected in a diagnostic sample, using assays and detection methods readily determined from the known art (e.g., immunological techniques, hybridization analysis, fluorescence imaging techniques, and/or radiation detection, etc.), as well as any assays and detection methods disclosed herein (e.g., immunoprecipitation, Western blot analysis, etc.). For example, a diagnostic sample of a subject may be assayed for cyclin E using an agent reactive with cyclin E. As used herein, “reactive” means the agent has affinity for, binds to, or is directed against cyclin E. The agent may include any of those described above. Preferably, the agent of the present invention is labeled with a detectable marker or label.

In one embodiment of the present invention, the agent reactive with cyclin E is an antibody. As used herein, the antibody of the present invention may be polyclonal or monoclonal. In addition, the antibody of the present invention may be produced by techniques well known to those skilled in the art. Polyclonal antibody, for example, may be produced by immunizing a mouse, rabbit, or rat with purified cyclin E. Monoclonal antibody then may be produced by removing the spleen from the immunized mouse, and fusing the spleen cells with myeloma cells to form a hybridoma which, when grown in culture, will produce a monoclonal antibody.

The antibodies used herein may be labeled with a detectable marker or label. Labeling of an antibody may be accomplished using one of a variety of labeling techniques, including peroxidase, chemiluminescent labels known in the art, and radioactive labels known in the art. The detectable marker or label of the present invention may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker or label may be a radioactive marker, including, for example, a radioisotope. The radioisotope may be any isotope that emits detectable radiation, such as ³⁵S, ³²p, ¹²⁵I, ³H, or ¹⁴C. Radioactivity emitted by the radioisotope can be detected by techniques well known in the art. For example, gamma emission from the radioisotope may be detected using gamma imaging techniques, particularly scintigraphic imaging. Preferably, the agent of the present invention is a high-affinity antibody labeled with a detectable marker or label.

Where the agent of the present invention is an antibody reactive with cyclin E, a diagnostic sample taken from the subject may be purified by passage through an affinity column which contains an anti-cyclin-E antibody as a ligand attached to a solid support, such as an insoluble organic polymer in the form of a bead, gel, or plate. The antibody attached to the solid support may be used in the form of a column. Examples of suitable solid supports include, without limitation, agarose, cellulose, dextran, polyacrylamide, polystyrene, sepharose, or other insoluble organic polymers. The antibody may be further attached to the solid support through a spacer molecule, if desired. Appropriate binding conditions (e.g., temperature, pH, and salt concentration) for ensuring binding of the agent and the antibody may be readily determined by the skilled artisan. In a preferred embodiment, the antibody is attached to a sepharose column, such as Sepharose 4B.

Where the agent is an antibody, a diagnostic sample of the subject may be assayed for cyclin E using binding studies that utilize one or more antibodies immunoreactive with cyclin E, along with standard immunological detection techniques. For example, the cyclin E protein eluted from the affinity column may be subjected to an ELISA assay, Western blot analysis, flow cytometry, or any other immunostaining method employing an antigen-antibody interaction. Preferably, the diagnostic sample is assayed for cyclin E using Western blotting.

Alternatively, a diagnostic sample of a subject may be assayed for cyclin E using hybridization analysis of nucleic acid extracted from the diagnostic sample taken from the subject. According to this method of the present invention, the hybridization analysis may be conducted using Northern blot analysis of mRNA. This method also may be conducted by performing a Southern blot analysis of DNA using one or more nucleic acid probes, which hybridize to nucleic acid encoding cyclin E. The nucleic acid probes may be prepared by a variety of techniques known to those skilled in the art, including, without limitation, the following: restriction enzyme digestion of cyclin E nucleic acid; and automated synthesis of oligonucleotides having sequences which correspond to selected portions of the nucleotide sequence of the cyclin E nucleic acid, using commercially-available oligonucleotide synthesizers, such as the Applied Biosystems Model 392 DNA/RNA synthesizer.

The nucleic acid probes used in the present invention may be DNA or RNA, and may vary in length from about 8 nucleotides to the entire length of the cyclin E nucleic acid. The cyclin E nucleic acid used in the probes may be derived from mammalian cyclin E. The nucleotide sequence for human cyclin E is known. Using this sequence as a probe, the skilled artisan could readily clone a corresponding cyclin E cDNA from other species. In addition, the nucleic acid probes of the present invention may be labeled with one or more detectable markers or labels. Labeling of the nucleic acid probes may be accomplished using one of a number of methods known in the art - e.g., nick translation, end labeling, fill-in end labeling, polynucleotide kinase exchange reaction, random priming, or SP6 polymerase (for riboprobe preparation)—along with one of a variety of labels—e.g., radioactive labels, such as ³⁵S, ³²P, or ³H, or nonradioactive labels, such as biotin, fluorescein (FITC), acridine, cholesterol, or carboxy-X-rhodamine (ROX). Combinations of two or more nucleic acid probes (or primers), corresponding to different or overlapping regions of the cyclin E nucleic acid, also may be used to assay a diagnostic sample for cyclin E, using, for example, PCR or RT-PCR.

The detection of cyclin E (or interaction between cyclin E and a parkin-associated complex) in the method of the present invention may be followed by an assay to measure or quantify the extent of cyclin E in a diagnostic sample of a subject. Such assays are well known to one of skill in the art, and may include immunohistochemistry/immunocytochemistry, flow cytometry, mass spectroscopy, Western blot analysis, or an ELISA for measuring amounts of cyclin E protein. For example, to use an immunohistochemistry assay, histological (paraffin-embedded) sections of tissue may be placed on slides, and then incubated with an antibody against cyclin E. The slides then may be incubated with a second antibody (against the primary antibody), which is tagged to a dye or other colorimetric system (e.g., a fluorochrome, a radioactive agent, or an agent having high electron-scanning capacity), to permit visualization of cyclin E present in the sections.

It is contemplated that the diagnostic sample in the present invention frequently will be assayed for cyclin E (or interaction between cyclin E and a parkin-associated complex) not by the subject or patient, nor by his/her consulting physician, but by a laboratory technician or other clinician. Accordingly, the method of the present invention further comprises providing to a subject's or patient's consulting physician a report of the results obtained upon assaying a diagnostic sample of the subject or patient for cyclin E.

The present invention further provides a method for assessing the efficacy of therapy to treat neurodegeneration in a subject or patient who has undergone or is undergoing treatment for neurodegeneration. The method of the present invention comprises assaying a diagnostic sample of the subject or patient for cyclin E, wherein a normal level of cyclin E in the diagnostic sample is indicative of successful therapy to treat neurodegeneration, and a level of cyclin E elevated above normal in the diagnostic sample is indicative of a need to continue therapy to treat neurodegeneration. In one embodiment of the present invention, a level of cyclin E elevated above normal is detected by detecting below-normal interaction between cyclin E and a parkin-associated complex. The neurodegeneration may be any of those described above, including sporadic Parkinson's disease, autosomal recessive early-onset Parkinson's disease, and neurodegeneration associated with glutamate excitotoxicity. The diagnostic sample may be assayed for cyclin E (or interaction between cyclin E and a parkin-associated complex) in vitro or in vivo. In addition, the diagnostic sample may be assayed for cyclin E (or interaction between cyclin E and a parkin-associated complex) using all of the various assays and methods of detection and quantification described above. This method of the present invention provides a means for monitoring the effectiveness of therapy to treat neurodegeneration by permitting the periodic assessment of levels of cyclin E (interaction between cyclin E and a parkin-associated complex) in a diagnostic sample taken from a subject or patient.

According to the method of the present invention, a diagnostic sample of a subject or patient may be assayed, and levels of cyclin E (interaction between cyclin E and a parkin-associated complex) may be assessed, at any time following the initiation of therapy to treat neurodegeneration. For example, levels of cyclin E (interaction between cyclin E and a parkin-associated complex) may be assessed while the subject or patient is still undergoing treatment for neurodegeneration. Where levels of cyclin E detected in an assayed diagnostic sample of the subject or patient continue to remain elevated above normal, a physician may choose to continue with the subject's or patient's treatment for the neurodegeneration. Where levels of cyclin E in an assayed diagnostic sample of the subject or patient decrease through successive assessments, it may be an indication that the treatment for neurodegeneration is working, and that treatment doses could be decreased or even ceased. Where levels of cyclin E in an assayed diagnostic sample of the subject or patient do not rapidly decrease through successive assessments, it may be an indication that the treatment for neurodegeneration is not working, and that treatment doses could be increased. Where cyclin E is no longer detected in an assayed diagnostic sample of a subject or patient at a level elevated above normal, a physician may conclude that the treatment for neurodegeneration has been successful, and that such treatment may cease.

It is within the confines of the present invention to assess levels of cyclin E (interaction between cyclin E and a parkin-associated complex) following completion of a subject's or patient's treatment for neurodegeneration, in order to determine whether the neurodegeneration has recurred in the subject or patient. Accordingly, an assessment of levels of cyclin E (interaction between cyclin E and a parkin-associated complex) in an assayed diagnostic sample may provide a convenient way to conduct follow-ups of patients who have been diagnosed with neurodegenerations. Furthermore, it is within the confines of the present invention to use assessed levels of cyclin E (interaction between cyclin E and a parkin-associated complex) in an assayed diagnostic sample as a clinical or pathologic staging tool, as a means of determining the extent of neurodegeneration in the subject or patient, and as a means of ascertaining appropriate treatment options.

A correlation exists, in general, between accumulation of cyclin E in neurons and Parkinson's disease. Therefore, it is also contemplated in the present invention that assaying a diagnostic sample of a subject for cyclin E may be a useful means of providing information concerning the prognosis of a subject or patient who has neurodegeneration. Accordingly, the present invention further provides a method for assessing the prognosis of a subject who has neurodegeneration, comprising assaying a diagnostic sample of the subject for cyclin E, wherein the subject's prognosis improves with a decreased level of cyclin E in the diagnostic sample, and the subject's prognosis worsens with an increased level of cyclin E in the diagnostic sample. In one embodiment of the present invention, the level of cyclin E elevated above normal is detected by detecting below-normal interaction between cyclin E and a parkin-associated complex. Suitable diagnostic samples, assays, and detection and quantification methods for use in the method of the present invention have already been described. This method of the present invention provides a means for determining the prognosis of a subject or patient diagnosed with neurodegeneration based upon the level of cyclin E, or interaction between cyclin E and a parkin-associated complex, in an assayed diagnostic sample of the subject or patient.

According to the method of the present invention, a diagnostic sample of a subject or patient may be assayed, and levels of cyclin E (or interaction between cyclin E and a parkin-associated complex) may be assessed, at any time during or following the diagnosis of neurodegeneration in the subject or patient. For example, levels of cyclin E (or interaction between cyclin E and a parkin-associated complex) in an assayed diagnostic sample may be assessed before the subject or patient undergoes treatment for neurodegeneration, in order to determine the subject's or patient's initial prognosis. Additionally, levels of cyclin E (or interaction between cyclin E and a parkin-associated complex) in an assayed diagnostic sample may be assessed while the subject or patient is undergoing treatment for neurodegeneration, in order to determine whether the subject's or patient's prognosis has become more or less favorable through the course of treatment.

For example, where the level of cyclin E detected in an assayed diagnostic sample of the subject or patient is, or continues to remain, significantly high, a physician may conclude that the subject's or patient's prognosis is unfavorable. Where the level of cyclin E in an assayed diagnostic sample of the subject or patient decreases through successive assessments, it may be an indication that the subject's or patient's prognosis is improving. Where the level of cyclin E in an assayed diagnostic sample of the subject or patient does not decrease significantly through successive assessments, it may be an indication that the subject's or patient's prognosis is not improving. Finally, where the level of cyclin E is low, or is normal, in a diagnostic sample of the subject or patient, a physician may conclude that the subject's or patient's prognosis is favorable.

The discovery that cyclin E can be detected in neurons displaying neurodegeneration provides a means of identifying patients with neurodegeneration, and presents the potential for commercial application in the form of a test for the diagnosis of neurodegeneration. The development of such a test could provide general screening procedures. Such procedures can assist in the early detection and diagnosis of neurodegeneration, and can provide a method for the follow-up of patients in whom a level of cyclin E elevated above normal has been detected.

Accordingly, the present invention further provides a kit for use as an assay of neurodegeneration, comprising a cyclin-E-specific agent and reagents suitable for detecting cyclin E. The cyclin-E-specific agent may be any agent reactive with cyclin E protein or nucleic acid, including a nucleic acid probe which hybridizes to nucleic acid encoding cyclin E, an antibody, and any of the agents described above. The agent may be used in any of the above-described assays or methods for detecting or quantifying levels of cyclin E. Preferably, the agent of the present invention is labeled with a detectable marker or label.

The present invention is described in the following Examples, which are set forth to aid in the understanding of the invention, and should not be construed to limit in any way the scope of the invention as defined in the claims which follow thereafter.

### EXAMPLES

### Example 1

### Expression Vectors, Cell Culture, and Antibodies

cDNAs for parkin, UbcH7, α-synuclein, and UbcH8 were PCR-amplified from a human liver cDNA library (Clontech), and cloned into the eukaryotic expression vectors, pCMS-EGFP (Clontech) or pcDNA3.1. Flag-parkin, T240R parkin, Flag-T240R parkin, and ΔUHD parkin were generated by PCR-mediated mutagenesis. A cDNA clone encoding PP2A/Bα was obtained from Research Genetics. HSel-10 constructs have been described (Wu et al., SEL-10 is an inhibitor of Notch signaling that targets Notch for ubiquitin-mediated protein degradation. Mol. Cell Biol., 21:7403-15, 2001). The integrity of all constructs was confirmed by automated sequencing. Recombinant baculoviruses expressing GST-tagged parkin were generated using the Baculogold system (Pharmingen), as per the manufacturer's instructions.

HeLa cells were maintained in Dulbecco's Modified Eagle Medium (Life Technologies), supplemented with 10% fetal bovine serum (Life Technologies), and heat-inactivated for 30 min at 50° C. Cells were transfected using Lipofectamine Plus (Life Technologies), incubated for 24-36 h, and treated as appropriate with 2.5 μM lactacystin (Sigma) for 16 h. Baculovirus expression and protein purifications were performed as described (Carrano et al., SKP2 is required for ubiquitin-mediated degradation of the CDK inhibitor p27. Nat. Cell Biol., 1: 193-99, 1999).

A parkin lentiviral vector was assembled by cloning the human parkin cDNA into the BamH1 and XhoI restriction enzyme sites of plasmid pTRIP GFP, and replacing the GFP gene (Zennou et al., The HIV-1 DNA flap stimulates HIV vector-mediated cell transduction in the brain. Nat. Biotechnol., 19:446-50, 2001). Parkin and control GFP viruses were produced by co-transfection of 293T cells with p8.91 and pHCMV-G; viral transduction of neuronal cultures was performed as described (Zennou et al., The HIV-1 DNA flap stimulates HIV vector-mediated cell transduction in the brain. Nat. Biotechnol., 19:446-50, 2001).

Parkin and cleaved-PARP polyclonal antibodies were obtained from Cell Signaling; α-Synuclein, UbH7, and Skp1 monoclonal antibodies were obtained from Transduction Labs; monoclonal rat antibody against DAT, and polyclonal rabbit antibodies against PP2A-Bα and GAD-65, were obtained from Chemicon; HA polyclonal antibody was obtained from Clontech; HRP-coupled Flag monoclonal antibody was obtained from Sigma; Myc polyclonal, cyclin D1 polyclonal, cyclin A1 polyclonal, and cyclin E monoclonal and polyclonal antibodies were obtained from Santa Cruz; Cul-1 and Rbx1 polyclonal antibodies were obtained from Zymed; and hSel-10 (69 kDa form) polyclonal antibody was obtained from Gentaur Molecular Products. Mouse monoclonal antibody, 2E10, against recombinant human parkin was generated using standard techniques (Ericson et al., Two critical periods of Sonic Hedgehog signaling required for the specification of motor neuron identity. Cell, 87:661-73, 1996). HRP-coupled goat-anti-mouse and goat-anti-rabbit secondary antibodies were obtained from Jackson Immunoresearch. Fluorescently-labeled secondary antibodies were obtained from Molecular Probes.

### Example 2

### Immunoprecipitation, Western Blot, and mRNA Analysis

Transiently transfected HeLa cells were suspended in lysis buffer (50 mM Tris (pH 7.6), 150 mM NaCl, 0.2% Triton X-100, and complete protease inhibitors (Sigma)), incubated for 60 min at 4° C., and cleared by centrifugation at 20,000 = g for 15 min at 4° C. Samples used for in vivo ubiquitination assays were suspended in lysis buffer supplemented with 2.5 mM N-ethyl maleimide (NEM). Lysates were subsequently quenched with 2.5 mM DTT for 20 min at 4° C. Immunoprecipitations and Western blotting were performed using standard techniques (Wu et al., SEL-10 is an inhibitor of Notch signaling that targets Notch for ubiquitin-mediated protein degradation. Mol. Cell Biol., 21:7403-15, 2001). Human brain tissue was obtained from the Columbia University Alzheimer's Disease Research Center Brain Bank. Quantitative real-time PCR was performed as described (Troy et al., Death in the balance: alternative participation of the caspase-2 and -9 pathways in neuronal death induced by nerve growth factor deprivation. J. Neurosci., 21:5007-16, 2001) using primers specific for cyclin E and β-actin.

### Example 3

### Banked Brain Tissue Analysis

ARPD mutant brain tissue was identified by genotyping of banked, early-onset PD brains for parkin mutations. One sample showed a 40-bp deletion in exon 3 (Δ438-477) in one allele of parkin, and a complete deletion of exon 8 in the other. Pathological analysis demonstrated depigmented substantia nigra without Lewy bodies (data not shown). Tissue was processed as below.

### Example 4

### Pull-Down and Ubiquitination Assays

Brain tissue (2 g per pulldown, maintained at 4° C.) was homogenized in 3× volume buffer (150 mM NaCl, 50 mM Tris (pH 7.6)), and centrifuged at 1,000×g for 15 min. 0.2% Triton X-100 was added to supernatants, and samples were centrifuged at 20,000×g for 20 min. Thereafter, samples were incubated with either parkin monoclonal-antibody-conjugated agarose beads (Pierce), or anti-Flag antibody-conjugated agarose beads (Sigma), along with recombinant Flag-hSel-10, for 2 h. Beads were washed five times with lysis buffer, and protein was eluted with LDS loading buffer (Life Technologies). In vitro ubiquitination assays were performed as described (Koepp et al., Phosphorylation-dependent ubiquitination of cyclin E by the SCFFbw7 ubiquitin ligase. Science, 294:173-77, 2001). Purified yeast E1, human UbcH7, ubiquitin, and ubiquitin aldehyde were obtained from Boston Biochem.

### Example 5

### Neuronal Assays

Cerebellar granule neurons from P6 mice were purified and transfected essentially as described (Scheiffele et al., Neuroligin expressed in nonneuronal cells triggers presynaptic development in contacting axons. Cell, 101:657-69, 2000). Dissociated cortical neurons from E16.5 mice were prepared and cultured as described (Scheiffele et al., Neuroligin expressed in nonneuronal cells triggers presynaptic development in contacting axons. Cell, 10 1:657-69, 2000). Primary midbrain cultures were prepared from E13.5 mouse embryos, as described (Hynes et al., Induction of midbrain dopaminergic neurons by Sonic hedgehog. Neuron, 15:35-44, 1995). Cells were treated for 24 h with or without kainate (250 or 500 μM as indicated; Sigma) or MPP⁺ (1 or 10 μM as indicated; Sigma), stained for 20 min with 0.5 μg/ml Hoechst 33342 (Sigma), and visualized by fluorescence microscopy. For granule cell assays, at least 100 cells in two 20× fields were scored in duplicate for GFP and/or Hoechst signal. For immunohistochemistry, cell cultures were washed twice with PBS, and fixed for 20 min with 4% (w/v) paraformaldehyde. Cells were blocked for 1 h, at room temperature, with 3% (v/v) goat serum in PBS, and then incubated overnight, at 4° C., with specific antibodies, as indicated. Cells were washed, incubated for 1 h at room temperature with appropriately-labeled secondary antibodies, and visualized by fluorescence microscopy. For midbrain culture experiments, DAT-specific immunoreactivity (pixels), and cytoplasmic parkin and cyclin E immunoreactivity (mean pixel density), were quantified in triplicate, across nine fields of view, at 20× using Image software (Scion).

SiRNAs were synthesized by Dharmacon Research, Inc., and duplexes were formed as per the manufacturer's instructions (parkin siRNA sequence: 5′ UUCCAAACCGG AUGAGUGGdTdT 3′; DAT siRNA sequence: 5′ GAGCGGGAGACCUGGAGCAdTdT 3′; SERT siRNA sequence: 5′ CUCCUGGAACACUGGCAACdTdT 3′). Cortical cultures were transfected using Lipofectamine 2000 reagent (Life Technologies); primary midbrain cultures were transfected using Transmessenger (Qiagen), as described (Krichevsky and Kosik, RNAi functions in cultured mammalian neurons. Proc. Natl Acad. Sci. USA, 99:11926-929, 2002).

Discussed below are results obtained by the inventors in connection with the experiments of Examples 1-5:

Parkin Interacts with HSel-10, an F-box/WD-repeat Domain Protein

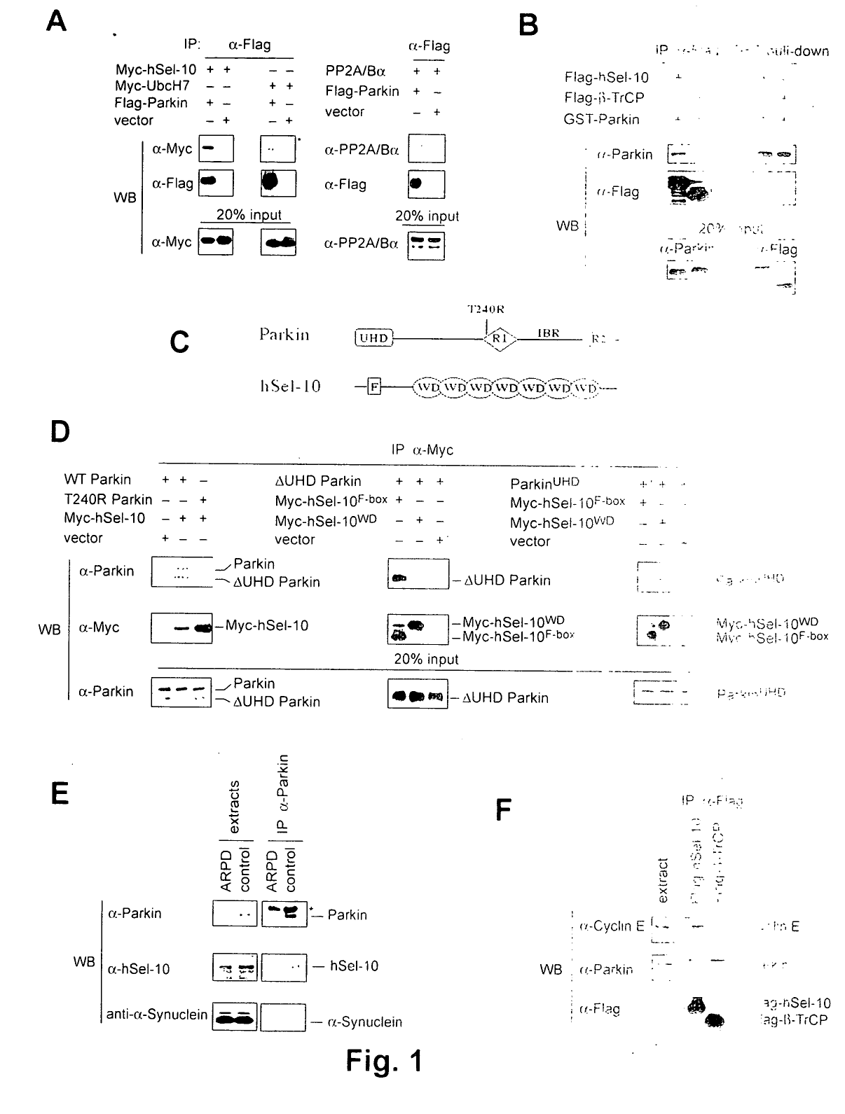

Epitope-tagged parkin and candidate interacting proteins were co-expressed in insect or HeLa cells; complexes were isolated by pull-down assays, and subsequently analyzed by Western blotting. Parkin was found to associate with hSel-10, an F-box/WD protein, in both the HeLa cell (FIG. 1A) and insect cell (FIG. 1B) systems. In contrast, parkin failed to associate with β-TRCP, a second F-box/WD protein (FIG. 1B). Parkin also failed to associate with several other WD-repeat-containing proteins (protein phosphatase 2A/Bα, the β subunit of heterotrimeric protein, and the Cockayne syndrome subunit A gene product) or F-box proteins (FIG. 1A and data not shown). Of note, hSel-10 and parkin are both predominantly cytoplasmic proteins enriched in adult brain (Koepp et al., Phosphorylation-dependent ubiquitination of cyclin E by the SCFFbw7 ubiquitin ligase. Science, 294:173-77, 2001). Parkin has also been reported to co-localize with Golgi apparatus and synaptic markers (Fallon et al., Parkin and CASK/LIN-2 associate via a PDZ-mediated interaction and are co-localized in lipid rafts and postsynaptic densities in brain. J. Biol. Chem., 25:25, 2001; Kubo et al., Parkin is associated with cellular vesicles. J. Neurochem., 78:42-54, 2001). Co-transfection of Flag-parkin and Myc-tagged UbcH7, followed by Flag immunoprecipitation, confirmed the previously-described association of parkin and the E2 UbcH7 (FIG. 1A) (Shimura et al., Familial Parkinson disease gene product, parkin, is a ubiquitin-protein ligase. Nat. Genet., 25:302-05, 2000), whereas no such interaction was observed with other E2s, including UbcH8 and Ubc6 (data not shown).

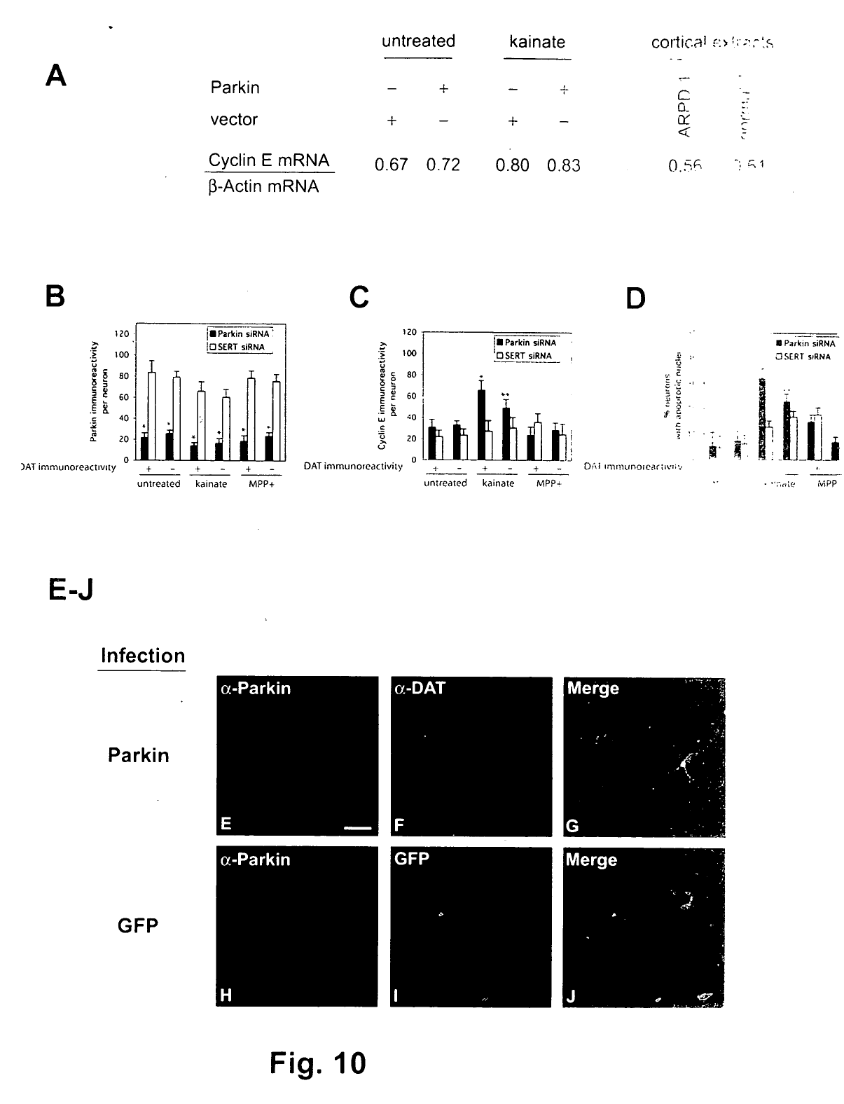

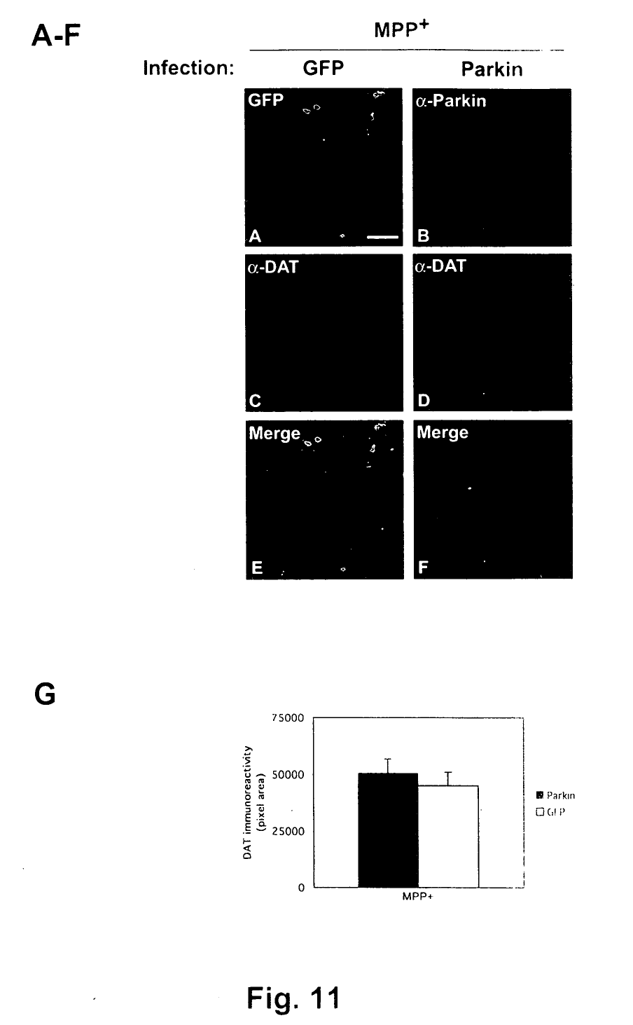

Deletion analysis of parkin and hSel-10 in transfected HeLa cells revealed that the carboxyl terminus of parkin, which includes the two RING finger domains, interacts specifically with the F-box of hSel-10 (FIGS. 1C and 1D). Furthermore, a missense mutation in parkin within the first RING finger (T240R; FIG. 1C), which leads to a familial ARPD syndrome (Shimura et al., Familial Parkinson disease gene product, parkin, is a ubiquitin-protein ligase. Nat. Genet., 25:302-05, 2000), abrogated the interaction with hSel-10 (FIG. 1D), consistent with the notion that this association is important for parkin function. A second interaction was apparent between either full-length or the amino-terminal ubiquitin homology domain (UHD) of parkin and the WD-repeat domain of hSel-10, but this interaction was not required for parkin-hSel-10 association (FIG. 1D and FIGS. 9-11).

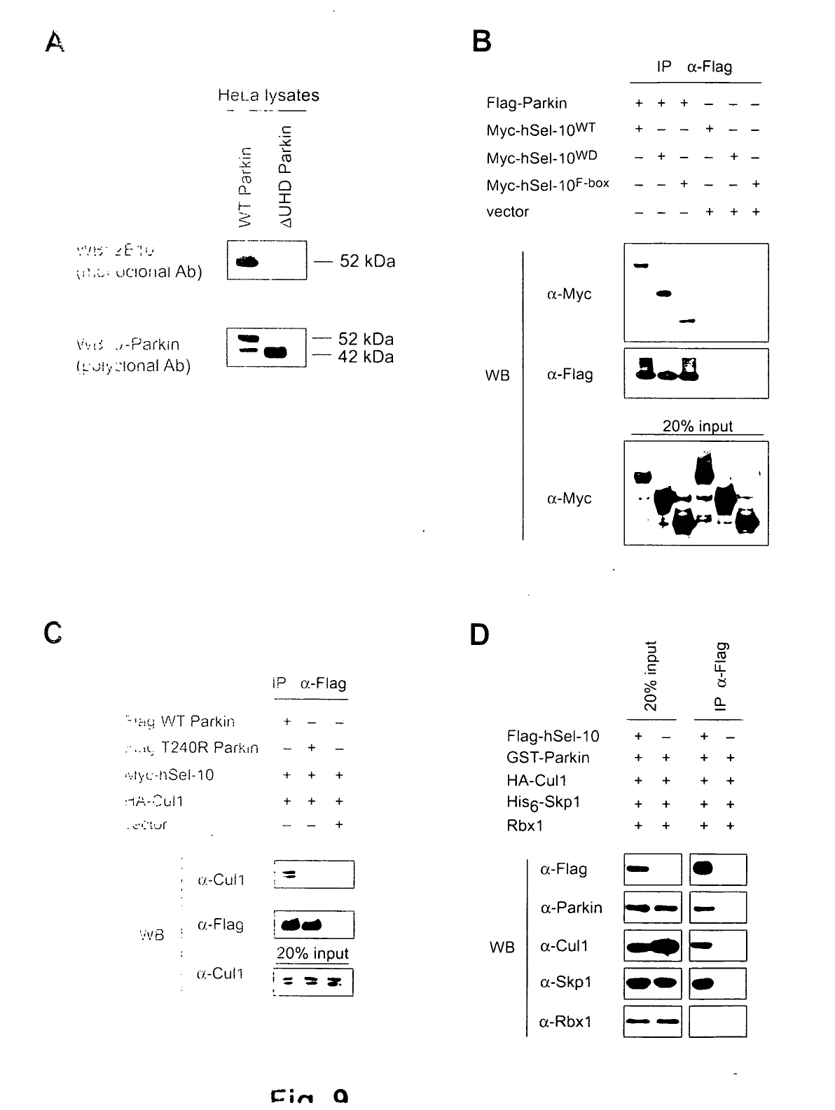

The inventors sought to confirm the interaction between parkin and hSel-10 in mammalian brain extracts. Immunoprecipitation of normal human frontal cortex extract with a monoclonal antibody specific for parkin protein (FIGS. 9-11), followed by Western blotting with a polyclonal antibody against hSel-10, indicated that parkin and hSel-10 are associated (FIG. 1E). In contrast, immunoprecipitation of an age-matched, parkin-deficient ARPD frontal cortex extract, with a Parkin-specific monoclonal antibody, failed to co-purify hSel-10. Parkin antibody immunoprecipitation of normal human frontal cortex extract (FIG. 1E) or transfected HeLa cell lysates (data not shown) failed to co-purify any form of α-synuclein. Purified Flag-hSel-10, when added to mouse cortex extract, associated with endogenous brain parkin in pull-down assays, whereas purified Flag-β-TrCP failed to do so (FIG. 1F). As previously reported (Strohmaier et al., Human F-box protein hCdc4 targets cyclin E for proteolysis and is mutated in a breast cancer cell line. Nature, 413:316-22, 2001), tagged hSel-10 effectively pulled down cyclin E as well (FIG. 1F).

HSel-10 Potentiates Parkin Ubiquitin Ligase Activity

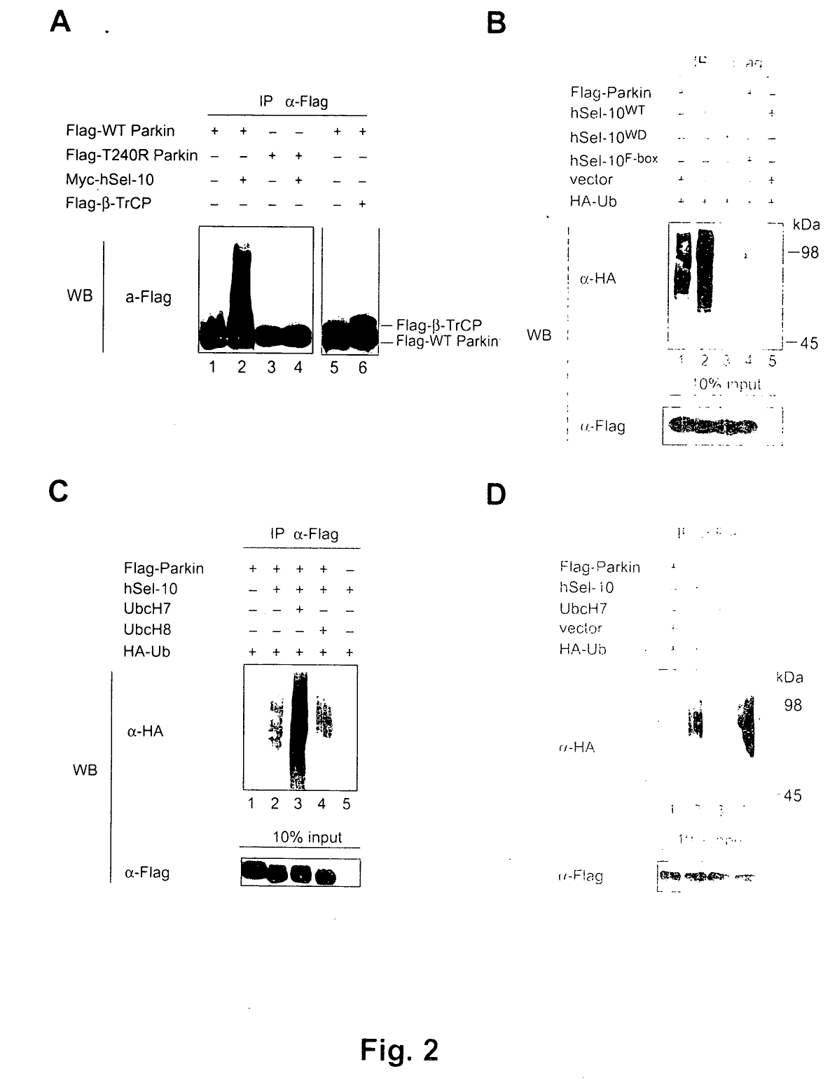

The inventors hypothesized that hSel-10 may be a component of a parkin-associated ubiquitin ligase complex, rather than a substrate. Consistent with this notion, the inventors did not observe parkin-dependent ubiquitination or proteolysis of hSel-10 (data not shown). Similar to several other RING-domain ubiquitin ligases, parkin auto-ubiquitinates (Zhang et al., Parkin functions as an E2-dependent ubiquitin-protein ligase and promotes the degradation of the synaptic vesicle-associated protein, CDCrel-1. Proc. Natl Acad. Sci. USA, 97:13354-359, 2000). The inventors examined whether hSel-10 overexpression modifies parkin ubiquitin ligase activity. Expression vectors encoding Flag-tagged wild-type or T240R (ARPD mutant) parkin, as well as either hSel-10 or β-TrCP, were transfected, along with hemagglutinin-(HA)-tagged ubiquitin, into HeLa cells. Flag immunoprecipitation from cell lysates, and subsequent Western blotting for the Flag tag, revealed a high-molecular-weight smear in lysates from cells transfected with wild-type (FIG. 2A, lane 1), but not T240R mutant parkin (FIG. 2A, lane 3), consistent with auto-ubiquitination of parkin. Indeed, Western blots of Flag immunoprecipitates for HA-ubiquitin (FIG. 2B, lane 1) again demonstrated a high-molecular-weight smear in lysates from wild-type parkin-transfected cells, confirming that these species are ubiquitinated derivatives of parkin.

Overexpression of hSel-10 dramatically potentiated the ubiquitin ligase activity of wild-type (FIG. 2A, lane 2 and FIG. 2B, lane 2), but not T240R ARPD mutant, parkin (FIG. 2A, lane 4). In contrast, overexpression of β-TrCP (FIG. 2A, lane 6) failed to potentiate parkin ubiquitin ligase activity. As HeLa cells express endogenous hSel-10 (Koepp et al., Phosphorylation-dependent ubiquitination of cyclin E by the SCFFbw7 ubiquitin ligase. Science, 294:173-77, 2001), the basal level of parkin ubiquitination in untransfected HeLa cells may be due to endogenous hSel-10 or a related activity. Therefore, the inventors co-transfected tagged parkin and ubiquitin expression constructs, as above, along with deletion mutants of hSel-10 (WD-repeat domain alone (WD) and F-box domain alone (F-box)) (Strohmaier et al., Human F-box protein hCdc4 targets cyclin E for proteolysis and is mutated in a breast cancer cell line. Nature, 413:316-22, 2001) that are thought to function in a dominant-negative manner, and to bind to wild-type parkin (FIGS. 1D and 9-11). Indeed, overexpression of these hSel-10 mutants inhibited parkin-mediated ubiquitination, indicating that parkin ubiquitin ligase activity requires hSel-10 or a related activity in vivo (FIG. 2B, lanes 3 and 4).

The inventors further investigated whether the E2 ubiquitin-conjugating enzyme, UbcH7, functions in the above parkin ubiquitination assay by co-transfecting a UbcH7 expression construct. Consistent with the protein interaction data (FIG. 1A), the inventors found that overexpression of UbcH7 (FIG. 2C, lane 3), but not UbcH8 (FIG. 2C, lane 4) or Ubc6 (data not shown), increased parkin-mediated ubiquitination. Furthermore, the enhancement of parkin-mediated ubiquitination, by UbcH7 overexpression, required co-expression of hSel-10 (FIG. 2D, lanes 3 and 4). Thus, parkin functions cooperatively with hSel-10 and UbcH7.

A Parkin-HSel-10-Cullin-1 Complex

HSel-10 has been shown to function in cell-cycle regulation within a modular, multiprotein E3 ubiquitin ligase complex, termed the SCF^{hSel-10} complex (for Skp1, Cullin, and F-box) (Patton et al., Combinatorial control in ubiquitin-dependent proteolysis: don't Skp the F-box hypothesis. Trends Genet., 14:236-43, 1998; Skowyra et al., F-box proteins are receptors that recruit phosphorylated substrates to the SCF ubiquitin-ligase complex. Cell, 91: 9-19, 1997), that includes Rbx1, a RING domain protein (Kamura et al., Rbx1, a component of the VHL tumor suppressor complex and SCF ubiquitin ligase. Science, 284:657-61, 1999; Skowyra et al., Reconstitution of G1 cyclin ubiquitination with complexes containing SCFGrr1 and Rbx1. Science, 284:662-65, 1999). Therefore, the inventors speculated that other characterized SCF^{hSel-10} components might be present in the parkin-hSel-10 complex.

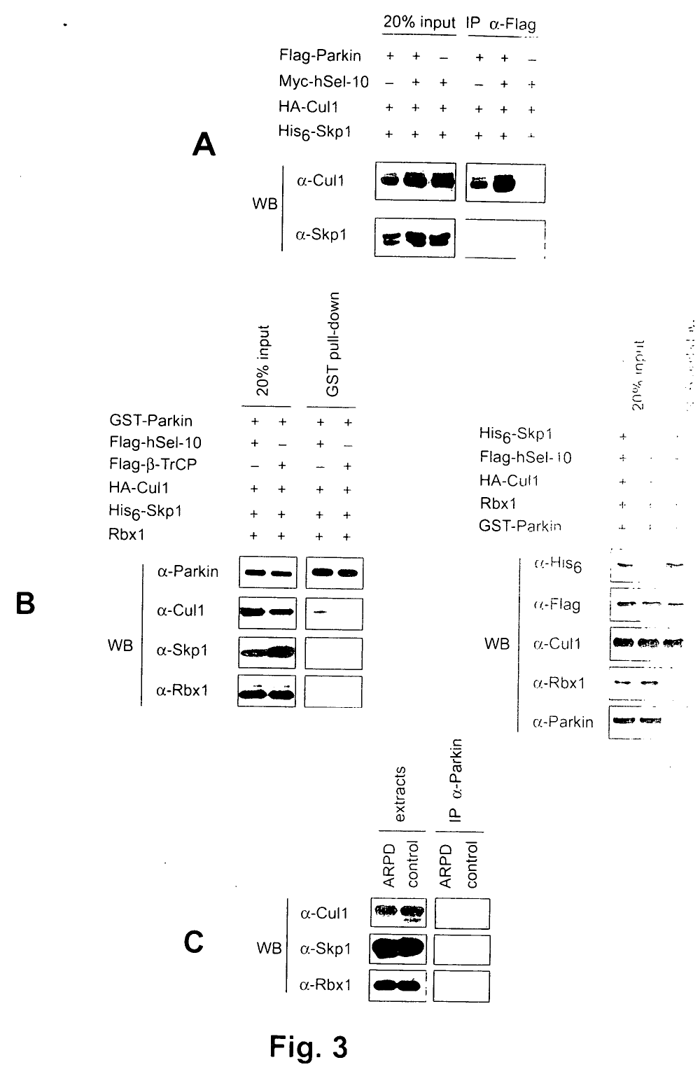

To investigate this possibility, the inventors co-expressed tagged parkin with tagged forms of Cul-1, Skp1, and Rbx1, in HeLa and insect cells. Subsequent pull-down assays revealed that parkin associates with Cul-1, but not Skp1 or Rbx1 (FIGS. 3A and 3B). The parkin-Cul-1 interaction appears to be modified by hSel-10, as the interaction is potentiated in HeLa cells that overexpress hSel-10 (FIG. 3A), and as parkin failed to associate with Cul-1 in insect cells in the absence of hSel-10 (FIG. 3B, left panel). Furthermore, the T240R ARPD parkin mutation attenuated the association of parkin with Cul-1 (FIGS. 9-11).

To investigate the relationship of the parkin-hSel-10 complex with the SCF^{hSel-10} complex, the inventors went on to perform a pull-down of His₆-Skp1 from the insect cell lysates. Tagged Skp1 co-purified Cul-1, hSel-10, and Rbx1 from insect cells (the SCF^{hSel-10} complex) (Koepp et al, Phosphorylation-dependent ubiquitination of cyclin E by the SCFFbw7 ubiquitin ligase. Science, 294:173-77, 2001; Strohmaier et al., Human F-box protein hCdc4 targets cyclin E for proteolysis and is mutated in a breast cancer cell line. Nature, 413:316-22, 2001), but not parkin (FIG. 3B, right panel). Flag-immunoprecipitation of hSel-10, as expected, co-precipitated parkin as well as all of the SCF components (FIGS. 9-11). Taken together, these data show that the parkin-hSel-10-Cul-1 complex is cooperative and distinct from the SCF^{hSel-10} complex.

The inventors next sought to confirm the presence of the parkin-hSel-10-Cul-1 complex in brain extracts. Immunoprecipitation of normal human frontal cortex brain extract (but not parkin-deficient ARPD frontal cortex extract), with a parkin-specific antibody, co-purified Cul-1, but not Skp1 or Rbx1 (FIG. 3C), consistent with the above data. Thus, parkin associates cooperatively with Cul-1 and hSel-10 in a novel complex that is distinct from SCF^{hSel-10}.

Ubiquitination of Cyclin E by Parkin

HSel-10 functions as an adaptor to recruit specific substrates for ubiquitination by the SCF^{hSel-10} complex, including cyclin E, a regulatory subunit of cyclin-dependent kinase 2 (CDK2) (Ekholm and Reed, Regulation of G(1) cyclin-dependent kinases in the mammalian cell cycle. Curr. Opin. Cell Biol., 12:676-84, 2000). Ubiquitination and degradation of phosphorylated cyclin E by the SCF^{hSel-10} complex underlies the regulation of cell-cycle entry into S phase. Interestingly, hSel-10 (Strohmaier et al., Human F-box protein hCdc4 targets cyclin E for proteolysis and is mutated in a breast cancer cell line. Nature, 413:316-22, 2001) is highly expressed in adult brain neurons, consistent with a role in post-mitotic cells. Of note, the accumulation of cyclins, including cyclin E, has been implicated in the regulation of apoptosis in post-mitotic neurons, as increased cyclin levels correlate with apoptosis (Verdaguer et al., Kainic acid-induced apoptosis in cerebellar granule neurons: an attempt at cell cycle re-entry. Neuroreport, 13:413-16, 2002), and cyclin-dependent kinase (CDK) inhibitors prevent such neuronal death (Copani et al., Activation of cell-cycle-associated proteins in neuronal death: a mandatory or dispensable path? Trends Neurosci., 24:25-31, 2001; Liu and Greene, Neuronal apoptosis at the G1/S cell cycle checkpoint. Cell Tissue Res., 305:217-28, 2001; Padmanabhan et al., Role of cell cycle regulatory proteins in cerebellar granule neuron apoptosis. J. Neurosci., 19:8747-56, 1999; Park et al., Cyclin-dependent kinases participate in death of neurons evoked by DNA-damaging agents. J. Cell Biol., 143:457-67, 1998). Furthermore, cyclin and CDK levels are increased in neurons in the course of several neurodegenerative disorders, such as PD (Chung et al., The role of the ubiquitin-proteasomal pathway in Parkinson's disease and other neurodegenerative disorders. Trends Neurosci., 24:S7-14, 2001; Husseman et al., Mitotic activation: a convergent mechanism for a cohort of neurodegenerative diseases. Neurobiol. Aging, 21:815-28, 2000).

The inventors hypothesized that hSel-10 may recruit cyclin E to the parkin-hSel-10-Cul-1 complex in post-mitotic neurons in a manner that is analogous to its role as an adaptor in SCF^{hSel-10}. Because hSel-10 is known to bind directly to cyclin E, it may recruit cyclin E and other substrates for modification by a parkin-hSel-10-Cul-1 E3 complex. Of note, cullins, including Cul-1, have been implicated directly in the ubiquitination of cyclin E (Dealy et al., Loss of Cull results in early embryonic lethality and dysregulation of cyclin E. Nat. Genet., 23:245-48, 1999; Singer et al., Cullin-3 targets cyclin E for ubiquitination and controls S phase in mammalian cells. Genes Dev., 13:2375-87, 1999). Thus, the inventors further hypothesized that the parkin-hSel-10-Cul-1 E3 complex may target cyclin E, and that parkin-associated ARPD may lead to toxic accumulation of this substrate.

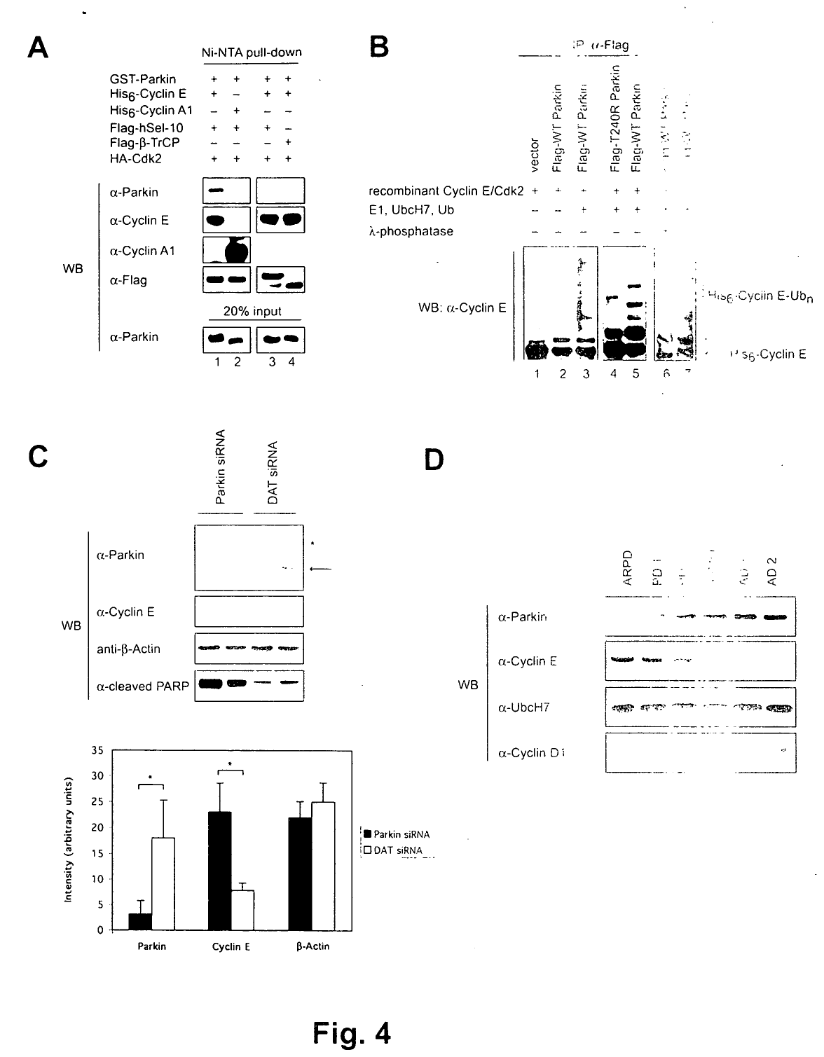

The inventors first tested the hypothesis that hSel-10 could recruit cyclin E to a parkin-associated complex. Insect cells were infected with baculoviruses encoding GST-parkin, Flag-hSel-10 (or Flag-β-TrCP), and His₆-cyclin E (or His₆-cyclin A1), and HA-CDK2 (which stabilizes phosphorylated forms of cyclin E that interact with hSel-10 (Clurman et al., Turnover of cyclin E by the ubiquitin-proteasome pathway is regulated by cdk2 binding and cyclin phosphorylation. Genes Dev., 10: 1979-90, 1996). Cell lysates were subsequently analyzed by pull-down assays and Western blotting. These studies confirmed that cyclin E (FIG. 4A, lane 1), but not cyclin A1 (lane 2), could be recruited to a parkin-associated complex by hSel-10 (FIG. 4A, lanes 1 and 3), but not by β-TrCP (FIG. 4A, lane 4).

The inventors also sought to determine whether a parkin-associated complex is able to ubiquitinate cyclin E substrate in vitro. The inventors found that a Flag-immunoprecipitated, wild-type parkin-associated complex (from lysates of HeLa cells transfected with Flag-parkin) could modify recombinant cyclin E/CDK2 substrate in the presence of other ubiquitination components in vitro (FIG. 4B, lanes 3, 5 and 7), whereas T240R ARPD mutant parkin complex failed to do so (FIG. 4B, lane 4). Furthermore, cyclin E ubiquitination appeared to be phosphorylation-dependent, as pre-treatment of the substrate with λ-phosphatase inhibited the modification (FIG. 4B, lanes 6 and 7).

Parkin Deficiency Potentiates the Accumulation of Cyclin E

The inventors hypothesized that parkin deficiency would potentiate the accumulation of cyclin E in primary neurons. Previous studies have indicated that primary neuronal cultures accumulate cyclin E in response to the glutamatergic excitotoxin, kainate (Padmanabhan et al., Role of cell cycle regulatory proteins in cerebellar granule neuron apoptosis. J. Neurosci., 19:8747-56, 1999; Verdaguer et al., Kainic acid-induced apoptosis in cerebellar granule neurons: an attempt at cell cycle re-entry. Neuroreport, 13:413-16, 2002), and the inventors confirmed this to be the case for primary cortical, cerebellar granule, and midbrain neuron cultures (see below, and data not shown).

To investigate the role of parkin in the accumulation of cyclin E, primary cortical cultures (prepared from embryonic day 16.5 (E16.5) embryos) were transfected with 25 nM parkin-specific or control (dopamine transporter-specific) short interfering RNAs (siRNAs) (Elbashir et al., Duplexes of 21-nucleotide RNAs mediate RNA interference in cultured mammalian cells. Nature, 411:494-98, 2001), and subsequently treated with kainate. Western-blot analysis of lysates from cultures transfected with control siRNA revealed readily detectable parkin protein expression (FIG. 4C), whereas lysates from parkin siRNA-transfected cultures displayed significantly reduced parkin expression. As predicted, parkin-deficient cultures displayed increased accumulation of cyclin E (FIG. 4C). Furthermore, such cultures displayed accumulation of cleaved poly (ADP-ribose) polymerase (cleaved-PARP), a marker of apoptosis.

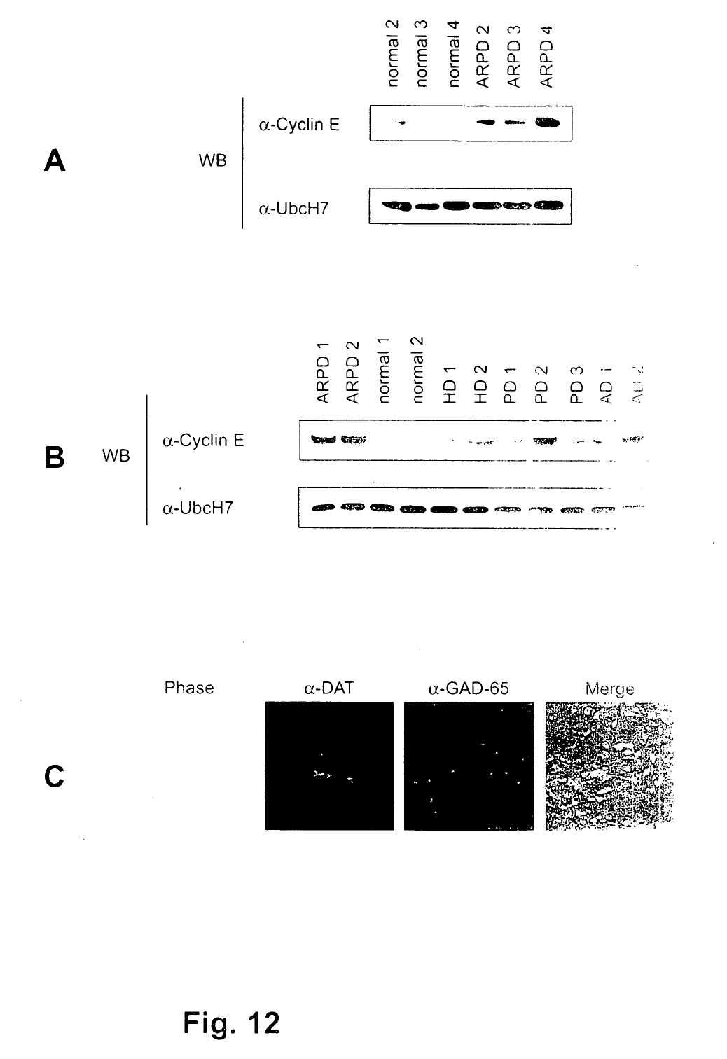

Parkin deficiency leads to neuronal loss in ARPD, and PD has been associated with apoptotic neuronal death (Burke and Kholodilov, Programmed cell death: does it play a role in Parkinson's disease? Ann. Neurol., 44:S126-33, 1998). Therefore, the inventors next investigated whether cyclin E is accumulated in extracts from Parkin-deficient human ARPD brain. Western blotting with a specific antibody demonstrated accumulation of cyclin E in substantia nigra from ARPD brain tissue extract, relative to age-matched control extract (FIG. 4D). In contrast, no accumulation was observed for three other proteins: cyclin D1, UbcH7, and a-synuclein (FIG. 4D, and data not shown). Similar accumulation of cyclin E was observed in frontal cortex extract from ARPD brain, and in cortical extracts from three independent ARPD cases, relative to three normal controls (FIG. 12).

Analysis of cyclin E MRNA by quantitative real-time PCR indicated that the accumulation of cyclin E protein was not accounted for by differences in cyclin E MRNA transcript levels (FIGS. 9-11). Analysis of cyclin E protein accumulation in substantia nigra extracts from sporadic PD and AD cases similarly demonstrated the accumulation of cyclin E in sporadic PD, but not sporadic AD, nigral extracts (FIG. 4D), consistent with the notion that cyclin E accumulation may be relevant to sporadic PD, as well as parkin-associated ARPD. Finally, analysis of frontal cortex extracts from sporadic PD (relative to AD, Huntington's disease, and normal control) brains revealed a variable degree of cyclin E accumulation (FIG. 12).

Parkin Overexpression Inhibits the Accumulation of Cyclin E

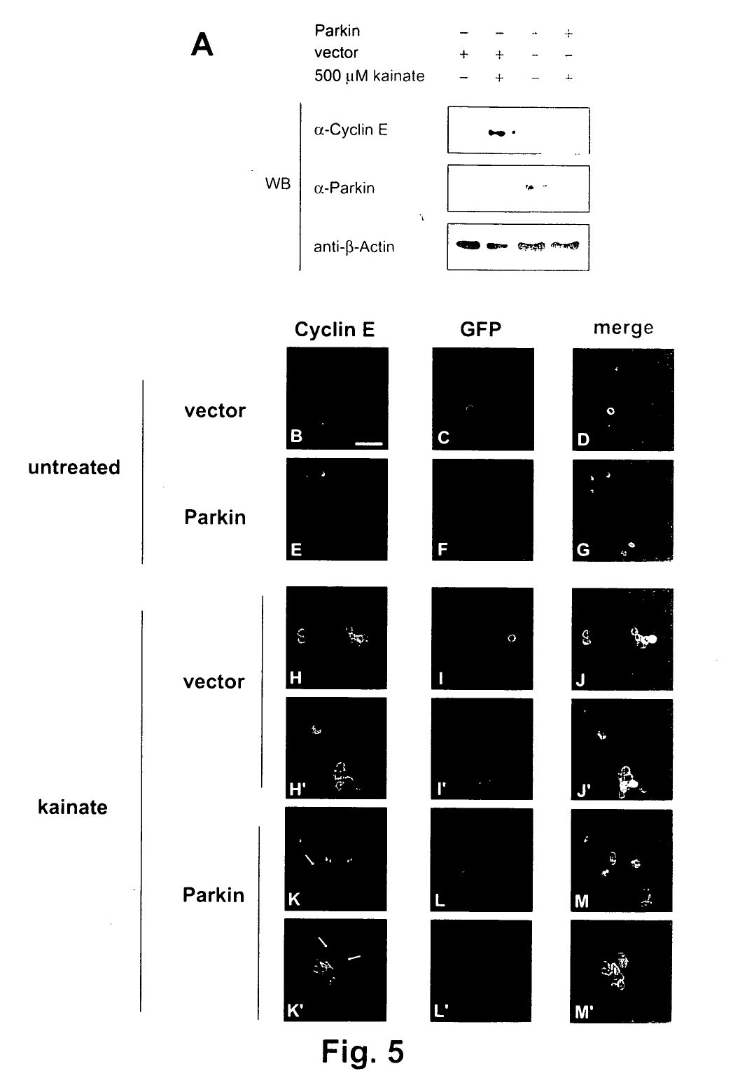

The inventors investigated the effect of parkin overexpression on kainate-induced apoptosis of cultured cerebellar granule cells, as these cells are readily purified to near homogeneity (Scheiffele et al., Neuroligin expressed in nonneuronal cells triggers presynaptic development in contacting axons. Cell, 101:657-69, 2000) and appear to be devoid of endogenous parkin expression (see FIG. 5A). Furthermore, cyclin E has been shown to accumulate with apoptosis in such cultures (Padmanabhan et al., Role of cell cycle regulatory proteins in cerebellar granule neuron apoptosis. J. Neurosci., 19:8747-56, 1999; Verdaguer et al., Kainic acid-induced apoptosis in cerebellar granule neurons: an attempt at cell cycle re-entry. Neuroreport, 13:413-16, 2002).

Granule neurons transfected with a bicistronic expression plasmid encoding wild-type parkin or vector alone (along with green fluorescent protein (GFP)) were treated with kainate (500 μM for 24 h). Subsequently, cultures were analyzed by Western blotting or immunofluorescence microscopy. As previously described, kainate treatment led to the accumulation of cyclin E in granule cell cultures (FIG. 5, panels A, B, E, H, and H′). Furthermore, overexpression of parkin significantly attenuated the accumulation of cyclin E (FIG. 5, panels H, H′, K, and K′). Analysis of cyclin E mRNA by quantitative real-time PCR indicated that the accumulation of cyclin E protein was not accounted for by differences in cyclin E MRNA (see FIGS. 9-11).

Parkin Overexpression Protects Post-Mitotic Neurons from Kainate-Mediated Excitotoxicity

Cell-cycle regulatory proteins have been implicated in the apoptotic death of post-mitotic cells. Cyclins, including cyclin E, accumulate in post-mitotic cells destined for apoptosis, whereas inhibitors of cyclin-dependent kinases block apoptosis (Copani et al., Activation of cell-cycle-associated proteins in neuronal death: a mandatory or dispensable path? Trends Neurosci., 24:25-31, 2001; Liu and Greene, Neuronal apoptosis at the G1/S cell cycle checkpoint. Cell Tissue Res., 305:217-28, 2001; Padmanabhan et al., Role of cell cycle regulatory proteins in cerebellar granule neuron apoptosis. J. Neurosci., 19:8747-56, 1999; Park et al., Cyclin-dependent kinases participate in death of neurons evoked by DNA-damaging agents. J. Cell Biol., 143:457-67, 1998). In contrast to cyclin E regulation at the G1/S cell-cycle checkpoint, little is known about the regulation of cyclin E accumulation in post-mitotic cells. Based on the above data, the inventors hypothesized that parkin may play a role in the regulation of cyclin E in the context of neuronal apoptosis, and that parkin overexpression would protect post-mitotic neurons from cell death.

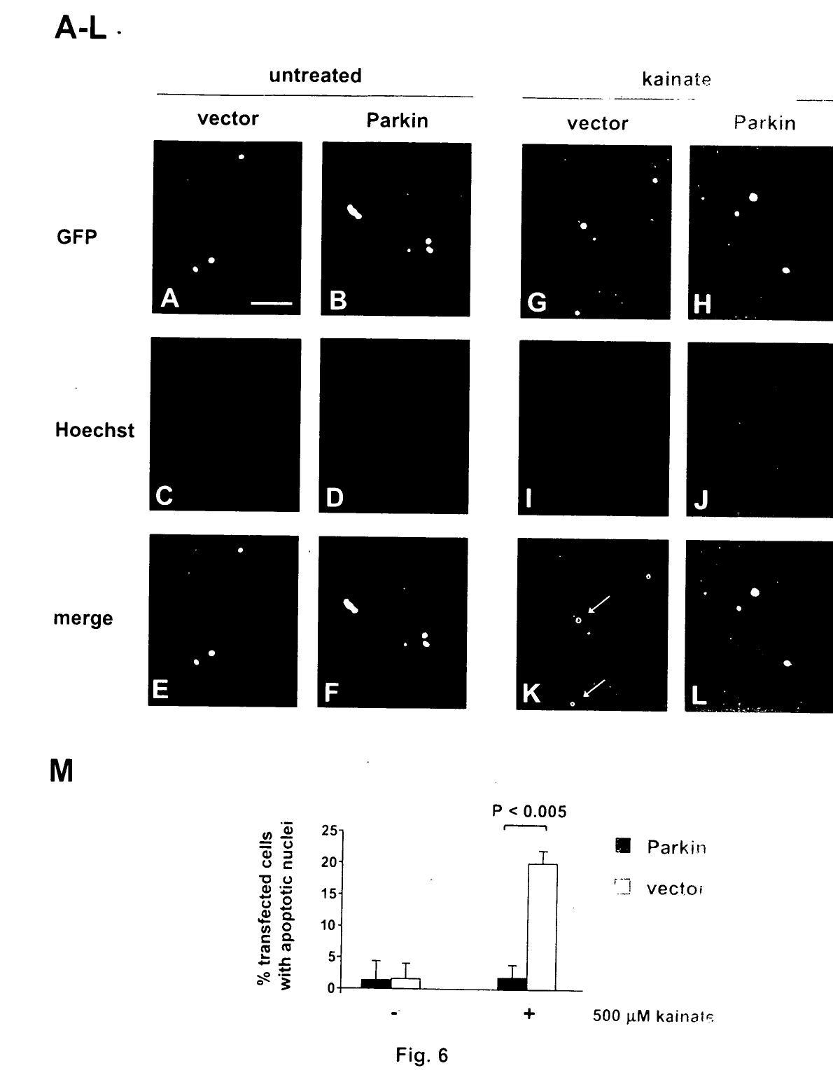

As described above, cyclin E is upregulated in the course of kainate-induced apoptosis of cultured cerebellar granule cells, and overexpression of parkin attenuates the accumulation of cyclin E. To investigate whether parkin overexpression would protect these cells from apoptosis, granule neurons were transfected and treated with kainate, as above. Apoptosis was quantified by visualization of condensed nuclei using Hoechst staining and fluorescence microscopy (FIG. 6). Neuronal cultures transfected with parkin showed significantly fewer apoptotic nuclei than vector-transfected cells (FIG. 6). Thus, parkin overexpression can protect post-mitotic neurons from toxin-mediated apoptosis, and this may be a consequence of inhibiting cyclin E accumulation.

Parkin and Dopiamine Neuron Survival

ARPD and sporadic PD lead to the relatively specific loss of dopamine neurons, although additional neuronal populations are affected to a variable extent. Furthermore, glutamate excitotoxicity has been implicated as a potential mechanism for dopamine neuron loss in PD ((Lang and Lozano, Parkinson's disease. First of two parts. N. Engl. J. Med., 339:1044-53, 1998; Olanow and Tatton, Etiology and pathogenesis of Parkinson's disease. Annu. Rev. Neurosci., 22 :123-44, 1999). As described above, parkin deficiency leads to increased cyclin E accumulation and the expression of apoptotic markers in the context of an excitotoxic insult to primary neurons, while parkin over-expression protects primary neurons. The inventors sought to extend these studies to primary dopamine neuron cultures.

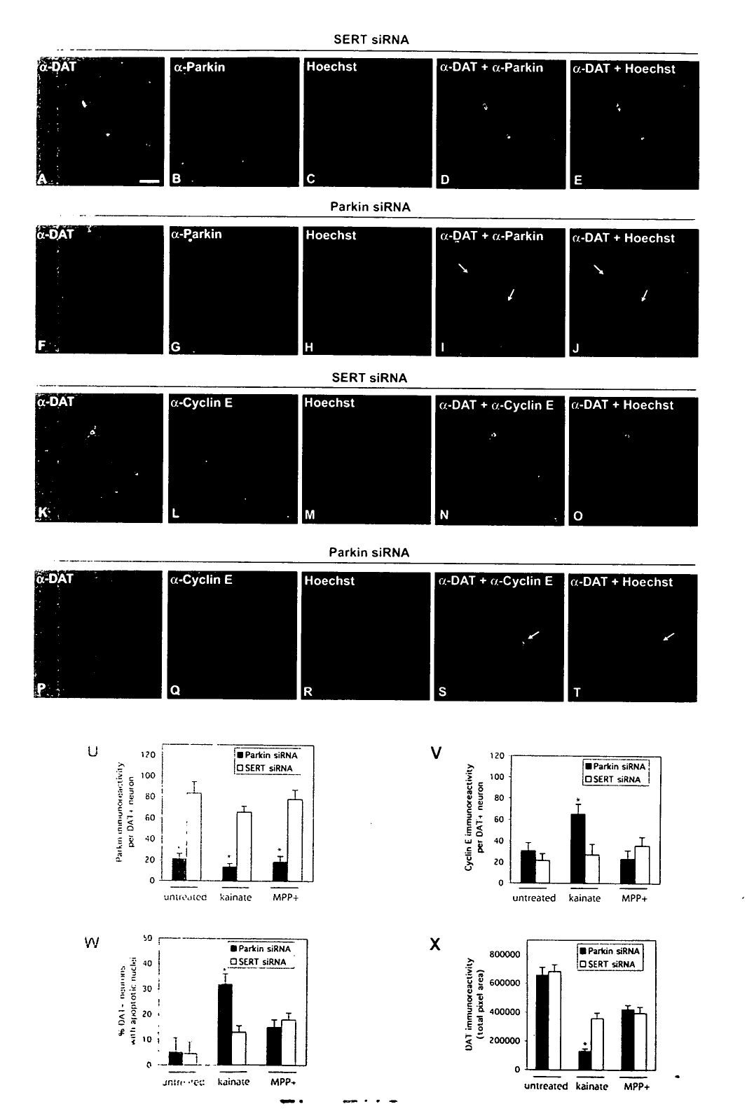

Embryonic day 13.5 (E13.5) primary culture midbrain dopamine neurons, identified by immunohistochemical staining for the dopamine transporter (DAT) (Nirenberg et al., The dopamine transporter is localized to dendritic and axonal plasma membranes of nigrostriatal dopaminergic neurons. J. Neurosci., 16:436-47, 1996), were transfected with parkin siRNA (FIG. 7, panels F-J and P-T) or control siRNA (FIG. 7, panels A-E and K—O), and subsequently exposed to kainate, as above. As predicted, parkin “knockdown” dopamine neurons displayed increased accumulation of cyclin E (FIG. 7, panel S) and increased apoptosis (FIG. 7, panels J and T), as compared with siRNA-treated cells. Furthermore, parkin-siRNA-treated midbrain cultures displayed decreased DAT immunoreactivity in cell bodies and processes in the presence of kainate, as compared with control siRNA-treated cells (FIG. 7, panel I), consistent with the increased sensitivity to kainate excitotoxicity. Parkin siRNA treatment alone, in the absence of kainate, failed to alter cyclin E or DAT immunoreactivity (FIG. 7, panels U—X); thus, parkin “knockdown” is not directly toxic, but appears to sensitize neurons to kainate excitotoxicity.

Parkin siRNA treatment failed to alter dopamine neuron sensitivity to 1-Methyl-4-phenylpyridinium (MPP⁺; 10 μM) at a toxin dose that (in control siRNA-treated cultures) led to a reduction in DAT immunoreactivity comparable to the kainate treatment (FIG. 7, panel X). The inventors further investigated the effect of parkin “knockdown” on the kainate sensitivity of DAT-negative neurons in midbrain cultures, which are primarily GABAergic (greater than 90%) (FIG. 12, in order to determine the specificity of parkin action. Parkin siRNA did sensitize DAT-negative neurons to kainate toxicity, but to a significantly lesser extent than it did the DAT-positive population, with respect to cyclin E induction and apoptosis (p<0.05 for both measures; see FIGS. 9-11). Thus, parkin deficiency appears to preferentially sensitize midbrain dopamine neurons to kainate excitotoxicity.

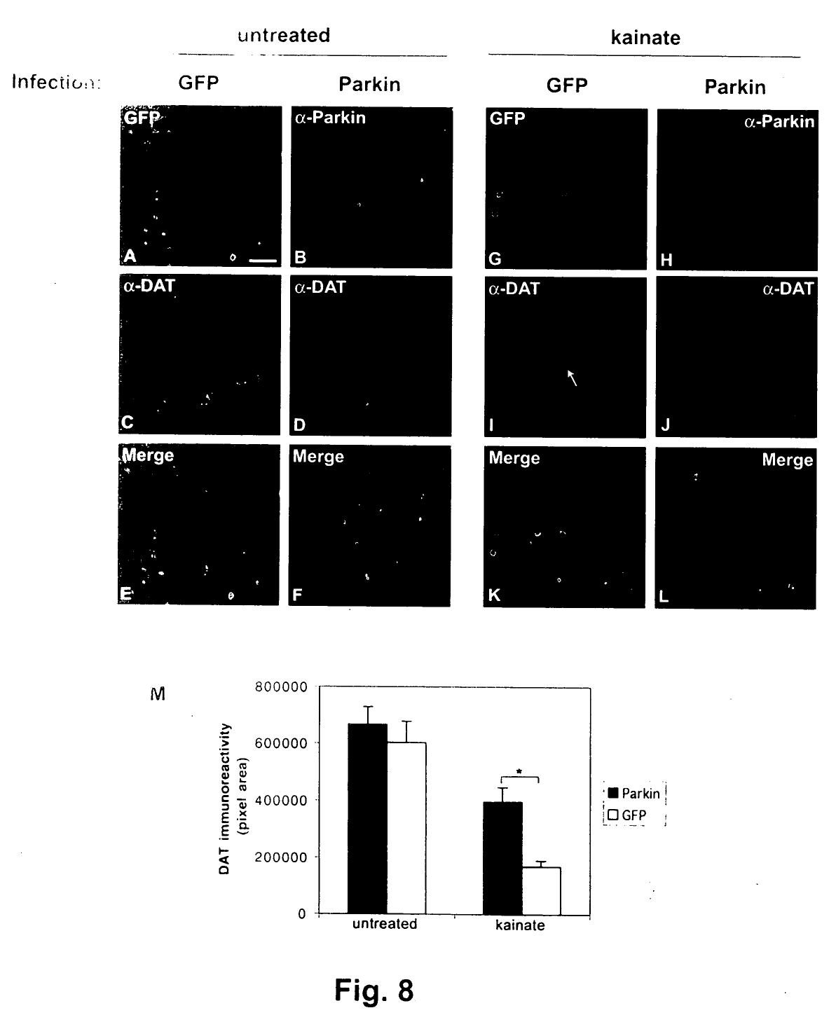

Overexpression of parkin using a lentiviral vector (in E13.5 midbrain dopamine neuron cultures) conferred robust protection of dopaminergic cell bodies and processes from 250 μM kainate, as quantified by DAT immunohistochemistry (FIG. 8), as compared to control lentivirus). Both parkin and control viruses infected over 90% of DAT-positive neurons (FIGS. 9-11, and data not shown). Parkin overexpression did not appear to alter sensitivity to MPP⁺ (see FIGS. 9-11). Furthermore, parkin overexpression did not alter DAT immunoreactivity in primary midbrain neuron cultures in the absence of toxin (FIG. 8, panel M).

As demonstrated above, parkin associates with hSel-10 and Cul-1 in a novel ubiquitin ligase complex. The parkin ubiquitin ligase complex functions in parkin auto-ubiquitination and in hetero-ubiquitination of cyclin E. The inventors also present evidence that parkin does appear to regulate cyclin E in the course of neuronal apoptosis, in dopamine neurons, and in ARPD tissue. The inventors hypothesize that, in addition to cyclin E, there are additional targets of parkin ubiquitination, as other characterized RING-finger-associated E3 complexes appear to target multiple diverse substrates (Joazeiro and Weissman, RING finger proteins: mediators of ubiquitin ligase activity. Cell, 102:549-52, 2000). For example, genetic and biochemical evidence implicate hSel-10 in the ubiquitination of Notch4 (Wu et al., SEL-10 is an inhibitor of Notch signaling that targets Notch for ubiquitin-mediated protein degradation. Mol. Cell Biol., 21:7403-15, 2001) and presenilin (Wu et al., Evidence for functional and physical association between Caenorhabditis elegans SEL-10, a Cdc4p-related protein, and SEL-12 presenilin. Proc. Natl Acad. Sci. USA, 95:15787-791, 1998), the latter of which is mutated in autosomal dominant forms of Alzheimer's disease. Thus, these represent additional candidates for activity of the parkin-associated complex.

SCF complexes are modular: for instance, Skp1 can interact with several F-box adaptor proteins, thereby generating functional diversity. It is of interest to determine whether parkin associates with adaptor proteins other than hSel-10 (although the inventors failed to detect an interaction with other F-box/WD-repeat proteins in the foregoing Examples), as such complexes would likely display different substrate specificities. This may explain the diverse targets that have been reported for parkin, including CDCrel-1 (Zhang et al., Parkin functions as an E2-dependent ubiquitin-protein ligase and promotes the degradation of the synaptic vesicle-associated protein, CDCrel-1. Proc. Natl Acad. Sci. USA, 97: 13354-359, 2000), synphilin-1 (Chung et al., Parkin ubiquitinates the alpha-synuclein-interacting protein, synphilin-1: implications for Lewy-body formation in Parkinson disease. Nat. Med., 7:1144-50, 2001), PAEL-R (Imai et al., An unfolded putative transmembrane polypeptide, which can lead to endoplasmic reticulum stress, is a substrate of Parkin. Cell, 105:891-02, 2001), and a modified form of α-synuclein (αSp22) (Shimura et al., Ubiquitination of a new form of alpha-synuclein by parkin from human brain: implications for Parkinson's disease. Science, 293:263-69, 2001). Recently, parkin has been reported to form a complex with the heat-shock protein, Hsp70, as well as CHIP, an Hsp70-associated protein with E3 activity, in SH-SY5Y cells that overexpress parkin (Imai et al., CHIP is associated with parkin, a gene responsible for familial Parkinson's disease, and enhances its ubiquitin ligase activity. Mol. Cell., 10:55-67, 2002). It remains to be determined whether SCF-like components play a role in this complex.

The inventors'data support the notion that there is both redundancy and specificity in the regulation of cyclin E (Koepp et al., Phosphorylation-dependent ubiquitination of cyclin E by the SCFFbw7 ubiquitin ligase. Science, 294:173-77, 2001; Winston et al., Culprits in the degradation of cyclin E apprehended. Genes Dev., 13:2751-57, 1999). Although cyclin E accumulation has been noted in the context of several apoptosis model systems, the mechanism of regulation of cyclin E in neuronal apoptosis has not previously been investigated. The data presented here suggest that parkin regulates the degradation of cyclin E in the context of neuronal apoptosis. Furthermore, as the parkin ubiquitin ligase complex targets cyclin E, this effect is likely to be direct.

Cyclin regulation, specifically regulation of cyclin E, has previously been implicated in kainate-excitotoxin-induced neuronal apoptosis (Padmanabhan et al., Role of cell cycle regulatory proteins in cerebellar granule neuron apoptosis. J. Neurosci., 19:8747-56, 1999; Verdaguer et al., Kainic acid-induced apoptosis in cerebellar granule neurons: an attempt at cell cycle re-entry. Neuroreport, 13:413-16, 2002). The inventors have shown herein that parkin overexpression protects dopamine neurons from kainate-mediated apoptosis, that parkin “knockdown” (using siRNA) sensitizes dopamine neurons to such excitotoxicity, and that this correlates with the accumulation of cyclin E. The inventors note that glutamate excitotoxicity has been implicated in sporadic PD (Olanow and Tatton, Etiology and pathogenesis of Parkinson's disease. Annu. Rev. Neurosci., 22:123-44, 1999; Schulz et al., The role of mitochondrial dysfunction and neuronal nitric oxide in animal models of neurodegenerative diseases. Mol. Cell. Biochem., 174:193-97, 1997), and that excitatory input ablation appears to protect dopamine neurons (Klein et al., The harlequin mouse mutation downregulates apoptosis-inducing factor. Nature, 419:367-74, 2002; Olanow and Tatton, Etiology and pathogenesis of Parkinson's disease. Annu. Rev. Neurosci., 22:123-44, 1999; Raina et al., Cyclin'toward dementia: cell cycle abnormalities and abortive oncogenesis in Alzheimer disease. J. Neurosci. Res., 61:128-33, 2000; Takada et al., Protection against dopaminergic nigrostriatal cell death by excitatory input ablation. Eur. J. Neurosci., 12:1771-80, 2000). Parkin deficiency appeared to preferentially sensitize midbrain dopamine neurons (versus midbrain GABAergic neurons) to kainate excitotoxicity, as may be the case in parkin-associated ARPD. Parkin overexpression did not appear to protect cultured primary dopamine neurons from MPP⁺ toxicity, and parkin knockdown (with siRNA) did not appear to sensitize dopamine neurons to MPP⁺ (at least under the conditions used here). These data suggest that different mechanisms may underlie kainate- and MPP⁺-mediated toxicity, and, indeed, it has been reported that MPP⁺ treatment induces non-apoptotic death in neuronal midbrain cultures (Lotharius et al., Distinct mechanisms underlie neurotoxin-mediated cell death in cultured dopaminergic neurons. J. Neurosci., 19:1284-93, 1999), in contrast with kainate. In a recent report (Petrucelli et al., Parkin protects against the toxicity associated with mutant alpha-synuclein: proteasome dysfunction selectively affects catecholaminergic neurons. Neuron, 36: 1007-19, 2002), parkin overexpression was found to protect primary midbrain catecholaminergic neurons from non-apoptotic death associated with the overexpression of mutant α-synuclein or proteasomal inhibition. The molecular mechanism of this protection appears to differ from that underlying the protection of primary neurons from excitotoxin-mediated apoptotic death, as described herein.

Finally, the protective role of parkin overexpression suggests a treatment approach for PD and other diseases that relate to glutamate excitotoxicity. Thus, an understanding of the parkin-associated ubiquitin ligase complex described herein, and its mechanism of action, can lead to novel diagnostic and therapeutic tools.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A parkin-associated complex, comprising parkin, hSel-10, and cullin-1.

2. The complex of claim 1, which has ubiquitin ligase activity.

3. The complex of claim 1, further comprising cyclin E.

4. The complex of claim 1, wherein parkin comprises the carboxyl terminus (residues 76-465) of parkin.

5. The complex of claim 4, wherein parkin comprises two RING finger domains.

6. The complex of claim 5, wherein parkin comprises amino acid residue T240.

7. A method for promoting ubiquitination of cyclin E in a post-mitotic neuron, comprising increasing activity of a parkin-associated complex in the neuron, wherein the parkin-associated complex comprises parkin, hSel-10, and cullin-1.

8. The method of claim 7, wherein the neuron is a dopamine neuron.

9. The method of claim 7, wherein activity of the parkin-associated complex is increased in the neuron by increasing the level of the complex in the neuron.

10. The method of claim 9, wherein activity of the parkin-associated complex is increased in the neuron by contacting the neuron with a parkin-associated agent, wherein the parkin-associated agent is selected from the group consisting of a parkin protein, a parkin mimetic, a modulator of parkin expression, and a modulator of parkin activity.

11. The method of claim 7, wherein the ubiquitination of cyclin E is promoted in vitro.

12. The method of claim 7, wherein the ubiquitination of cyclin E is promoted in vivo in a subject.

13. The method of claim 12, wherein the ubiquitination of cyclin E is promoted in vivo in a subject by administering a parkin-associated agent to the subject.

14. The method of claim 13, wherein the subject is a human.

15. The method of claim 14, wherein the human has neurodegeneration.

16. The method of claim 15, wherein the neurodegeneration is selected from the group consisting of Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease.

17. The method of claim 16, wherein the neurodegeneration is sporadic Parkinson's disease or autosomal recessive early-onset Parkinson's disease.

18. The method of claim 15, wherein the neurodegeneration is associated with glutamate excitotoxicity.

19. The method of claim 13, wherein parkin is administered to the subject orally, intradermally, intramuscularly, intraperitoneally, intravenously, or subcutaneously.

20. A therapeutic composition, comprising:
(a) a nucleic acid encoding a parkin-associated agent;
(b) a lentiviral vector; and
(c) optionally, a pharmaceutically-acceptable carrier;
wherein the parkin-associated agent is selected from the group consisting of a parkin protein, a parkin mimetic, a modulator of parkin expression, and a modulator of parkin activity.

21. A method for treating neurodegeneration in a subject in need of treatment, comprising administering to the subject the therapeutic composition of claim 20, in an amount effective to treat the neurodegeneration in the subject.

22. The method of claim 21, wherein the neurodegeneration is sporadic Parkinson's disease or autosomal recessive early-onset Parkinson's disease.

23. Use of the therapeutic composition of claim 20 in an animal model of Parkinson's disease.

24. A method for identifying an agent which interacts with a parkin-associated complex, comprising the steps of:
(a) contacting a candidate agent with the complex, in the presence of cyclin E; and
(b) assessing the ability of the candidate agent to enhance interaction between the complex and cyclin E;
wherein the parkin-associated complex comprises parkin, hSel-10, and cullin-1.

25. The method of claim 24, further comprising the steps of:
(c) contacting the candidate agent with at least one post-mitotic neuron containing cyclin E; and
(d) determining if the agent has an effect on a cyclin-E-associated biological event in the at least one neuron.

26. The method of claim 25, wherein the cyclin-E-associated biological event is selected from the group consisting of accumulation of cyclin E, ubiquitination of cyclin E, apoptosis, and cyclin-E-associated neurodegeneration.

27. The agent identified by the method of claim 24.

28. The agent identified by the method of claim 25.

29. A method for protecting at least one post-mitotic neuron from excitotoxicity, comprising contacting the at least one neuron with an amount of the agent of claim 28 effective to protect the at least one neuron from excitotoxicity.

30. The method of claim 29, wherein the neuron is a dopamine neuron.

31. A method for treating or preventing neurodegeneration in a subject, comprising administering to the subject an amount of the agent of claim 28 effective to treat or prevent the neurodegeneration in the subject.

32. The method of claim 31, wherein the neurodegeneration is selected from the group consisting of Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease.

33. The method of claim 32, wherein the neurodegeneration is sporadic Parkinson's disease or autosomal recessive early-onset Parkinson's disease.

34. The method of claim 31, wherein the neurodegeneration is associated with glutamate excitotoxicity.

35. A method for decreasing cyclin E in at least one post-mitotic neuron, comprising contacting the at least one neuron with a parkin-associated agent, in an amount effective to decrease cyclin E in the neuron, wherein the parkin-associated agent is selected from the group consisting of a parkin protein, a parkin mimetic, a modulator of parkin expression, and a modulator of parkin activity.

36. The method of claim 35, wherein cyclin E is decreased in the at least one neuron by decreasing the level of cyclin E in the neuron or by decreasing accumulation of cyclin E in the neuron.

37. The method of claim 35, wherein the neuron is selected from the group consisting of a cerebellar granule neuron, a cortical neuron, and a substantia nigra neuron.

38. The method of claim 35, wherein the neuron is damaged.

39. The method of claim 38, wherein the damage results from excitotoxicity.

40. The method of claim 35, wherein the contacting is effected in vitro.

41. The method of claim 35, wherein the contacting is effected in vivo in a subject.

42. The method of claim 41, wherein the contacting is effected in vivo in a subject by administering the parkin-associated agent to the subject.

43. The method of claim 42, wherein the subject is a human.

44. The method of claim 43, wherein the human has neurodegeneration.

45. A method for protecting at least one post-mitotic neuron from excitotoxicity, comprising contacting the at least one neuron with a parkin-associated agent, in an amount effective to protect the neuron from excitotoxicity, wherein the parkin-associated agent is selected from the group consisting of a parkin protein, a parkin mimetic, a modulator of parkin expression, and a modulator of parkin activity.

46. The method of claim 45, wherein the neuron is a dopamine neuron.

47. The method of claim 45, wherein the neuron is a cerebellar granule cell.

48. The method of claim 45, wherein the excitotoxicity is glutamate excitotoxicity.

49. The method of claim 48, wherein the glutamate excitotoxicity is kainate-mediated excitotoxicity.

50. The method of claim 45, wherein protection from excitotoxicity results in protection from apoptosis.

51. The method of claim 45, wherein the contacting is effected in vitro.

52. The method of claim 45, wherein the contacting is effected in vivo in a subject.

53. The method of claim 52, wherein the contacting is effected in vivo in a subject by administering the parkin-associated agent to the subject.

54. Use of a parkin-associated agent to protect a post-mitotic neuron from excitotoxicity, wherein the neuron is contacted with an amount of parkin-associated agent effective to protect the neuron from excitotoxicity, and wherein the parkin-associated agent is selected from the group consisting of a parkin protein, a parkin mimetic, a modulator of parkin expression, and a modulator of parkin activity.

55. A method for determining whether a subject has neurodegeneration, comprising assaying a diagnostic sample of the subject for cyclin E, wherein detection of a cyclin E level elevated above normal is diagnostic of neurodegeneration in the subject.

56. The method of claim 55, wherein the neurodegeneration is sporadic Parkinson's disease or autosomal recessive early-onset Parkinson's disease.

57. The method of claim 55, wherein the diagnostic sample is a sample from the frontal cortex, midbrain, or substantia nigra of the subject.

58. The method of claim 55, wherein the diagnostic sample is assayed using an agent reactive with cyclin E.

59. The method of claim 58, wherein the agent is labeled with a detectable marker.

60. The method of claim 58, wherein the agent is an antibody.

61. The method of claim 55, wherein the diagnostic sample is assayed using at least one nucleic acid probe which hybridizes to nucleic acid encoding cyclin E.

62. The method of claim 61, wherein the nucleic acid probe is labeled with a detectable marker.

63. A method for assessing the efficacy of therapy to treat neurodegeneration in a subject who has undergone or is undergoing treatment for neurodegeneration, comprising assaying a diagnostic sample of the subject for cyclin E, wherein a normal level of cyclin E in the diagnostic sample is indicative of successful therapy to treat neurodegeneration, and a level of cyclin E elevated above normal in the diagnostic sample is indicative of a need to continue therapy to treat neurodegeneration.

64. A method for assessing the prognosis of a subject who has neurodegeneration, comprising assaying a diagnostic sample of the subject for cyclin E, wherein the subject's prognosis improves with a decreased level of cyclin E in the diagnostic sample, and the subject's prognosis worsens with an increased level of cyclin E in the diagnostic sample.

65. A kit for use in detecting neurodegeneration, comprising:
(a) a cyclin-E-specific agent; and
(b) reagents suitable for detecting cyclin E;
wherein the cyclin-E-specific agent is selected from the group consisting of an agent reactive with cyclin E and a nucleic acid probe which hybridizes to nucleic acid encoding cyclin E.

66. The kit of claim 65, wherein the cyclin-E-specific agent is labeled with a detectable marker.

67. The kit of claim 65, wherein the agent reactive with cyclin E is an antibody.
